SEQUENCE LISTING (8 sequences)

SEQ ID NO: 1 - protein, 448 aa, Thermotoga maritima
  Deposit number gi|4980806|gb|AAD3 5394.1|AE001712_7, alpha-L-fucosidase
MISMKPRYKPDWESLREHTVPKWFDKAKFGIFIHWGIYSVPGWATPTGELGKVPMDAWFFQNPYAEWYENSLRIKESPTWEYHVKTYGENFEYEKFADLFTAEKWDPQEWADLFKKAGAKYVIPTTKHHDGFCLWGTKYTDFNSVKRGPKRDLVGDLAKAVREAGLRFGVYYSGGLDWRFTTEPIRYPEDLSYIRPNTYEYADYAYKQVMELVDLYLPDVLWNDMGWPEKGKEDLKYLFAYYYNKHPEGSVNDRWGVPHWDFKTAEYHVNYPGDLPGYKWEFTRGIGLSFGYNRNEGPEHMLSVEQLVYTLVDVVSKGGNLLLNVGPKGDGTIPDLQKERLLGLGEWLRKYGDAIYGTSVWERCCAKTEDGTEIRFTRKCNRIFVIFLGIPTGEKIVIEDLNLSAGTVRHFLTGERLSFKNVGKNLEITVPKKLLETDSITLVLEAVE

SEQ ID NO: 2 - protein, 403 aa, Sulfolobus solfataricus
  Deposit number gi|13816464|gb|AA K43159.1| Alpha-fucosidase C-terminal fragment (fucA1) [Sulfolobus solfataricus P2]
MFTGENWDPYEWAKVFKKSGAKFVVLVAEHHDGFALWESNYTRWCATKIGPKRDIVRELKEAVEGQGLIFGISYHRAEHWWFFDQGMKIESDVKDPRYLDLYGPAQSASLNPRDPPSLDNVQPNDEFLMDWLLRIVEAVEKYRPWLVYFDWWIANPSFQPYLKAFASYYYNRSYKWGIEPVIIYKQGAFGEGTAIPDLAERGTIKNVYPSTWLADTSIDYKSWGYIKDAEYKLPSVILSHLGDVVSKNGVFLLNIGPKADGTIPEEAKRILLDVGDWLNVNGEAIFGSKPWRVYGEGPSGINEGGFFTERKITLGYQDVRYTVKDYYPRQRHIYAILFGKPKEITLRSFMKNLKLIEEAVIVDVSRLDGKGKLEWSLSDEGLKIKIEEVIRAPLVIRVILDYR

SEQ ID NO: 3 - protein, 1959 aa, Bifidobacterium bifidum
  Deposit numbergi|34451973|gb|AAQ 72464.1| alpha-fucosidase [Bifidobacterium bifidum JCM 1254]
MKHRAMSSRLMPLVASCATVGMLLAGLPVSAVAVGTTRAAASDASSSTTATITPSADTTLQTWTSEKNSSMASKPYIGTLQGPSQGVFGEKFESTDAADTTDLKTGLLTFDLSAYDHAPDSATFEMTYLGYRGNPTATDTDTIKVTPVDTTVCTNNATDCGANVATGATKPKFSINDSSFVAESKPFEYGTTVYTGDAITVVPANTKKVTVDVTEIVRQQFAEGKKVITLAVGETKKTEVRFASSEGTTSLNGATADMAPKLTVSVSTKDDLKPSADTTLQAWASEKNEKKNTAAYVGALQPEGDYGDFGEKFKSTDVHDVTDAKMGLMTFDLSDYTAAPEHSILTLTYLGYAGADKTATATDKVKVVAVDTSRCTGTAPCDTNNATWANRPDFEVTDTTKTATSHAFAYGSKKYSDGMTVESGNAKKVLLDVSDVIKAEFAKFSAGATEKKITLALGELNKSDMRFGSKEVTSLTGATEAMQPTLSVTKKPKAYTLSIEGPTKVKYQKGEAFDKAGLVVKATSTADGTVKTLTEGNGEDNYTIDTSAFDSASIGVYPVTVKYNKDPEIAASFNAYVIASVEDGGDGDTSKDDWLWYKQPASQTDATATAGGNYGNPDNNRWQQTTLPFGNGKIGGTVWGEVSRERVTFNEETLWTGGPGSSTSYNGGNNETKGQNGATLRALNKQLANGAETVNPGNLTGGENAAEQGNYLNWGDIYLDYGFNDTTVTEYRRDLNLSKGKADVTFKHDGVTYTREYFASNPDNVMVARLTASKAGKLNFNVSMPTNTNYSKTGETTTVKGDTLTVKGALGNNGLLYNSQIKVVLDNGEGTLSEGSDGASLKVSDAKAVTLYIAAATDYKQKYPSYRTGETAAEVNTRVAKVVQDAANKGYTAVKKAHIDDHSAIYDRVKIDLGQSGHSSDGAVATDALLKAYQRGSATTAQKRELETLVYKYGRYLTIGSSRENSQLPSNLQGIWSVTAGDNAHGNTPWGSDFHMNVNLQMNYWPTYSANMGELAEPLIEYVEGLVKPGRVTAKVYAGAETTNPETTPIGEGEGYMAHTENTAYGWTAPGQSFSWGWSPAAVPWILQNVYEAYEYSGDPALLDRVYALLKEESHFYVNYMLHKAGSSSGDRLTTGVAYSPEQGPLGTDGNTYESSLVWQMLNDAIEAAKAKGDPDGLVGNTTDCSADNWAKNDSGNFTDANANRSWSCAKSLLKPIEVGDSGQIKEWYFEGALGKKKDGSTISGYQADNQHRHMSHLLGLFPGDLITIDNSEYMDAAKTSLRYRCFKGNVLQSNTGWAIGQRINSWARTGDGNTTYQLVELQLKNAMYANLFDYHAPFQIDGNFGNTSGVDEMLLQSNSTFTDTAGKKYVNYTNILPALPDAWAGGSVSGLVARGNFTVGTTWKNGKATEVRLTSNKGKQAAVKITAGGAQNYEVKNGDTAVNAKVVTNADGASLLVFDTTAGTTYTITKKASANVPVTGVTVTGANTATAGDTVTLTATVAPANATDKSVTWSTSDAAVATVNANGVVTTKKAGKVTITATSNGDKTKFGSIEITVSAATVPVTSVTVAGDAAMTVDGEQTLTATVAPATATDKTVTWKSSDATVATVDANGKVVAKKAGEVTITATAGGVSGTLKITVSDKAPTVIPVQSVTVTGKQELVEGASTTLTATVAPADATDKTVTWKSSDESVATVDKDGVVTAKKAGTVTITATAGGVSGTLHITVTAKPVETVPVTSVEVTVEAGTTVSVGKTLQATATVKPGNATNKKVTWKSSDESIATVDANGVITAKKAGKVVITATSTDGTDKSGSVEITVVDETKPTPDHKSVKADTGDVTAGKTGTVTEPKDVAGWKSRSIIKQGKLGKAEIADGTLVYAAGDKTGDDSFVVQYTMADGTVIDVTYSVTVKAAETGKNDGDGKGDGVAKTGAAVGALAGLGLMLLAVGVSVVMIRRKHSA

SEQ ID NO: 4 - protein, 1493 aa, Bifidobacterium bifidum
  gi|242345155|dbj|BAH80310.1| alpha-L-fucosidase [Bifidobacterium bifidum JCM 1254]
MLHTASRGCSRSWLRRLTALIAVSALAFVALPNVAVAADPMEYLDVSFGGTFAADTYTTGGDEVAKGPVTKHGSIPTKLDGGGITLAGGTNGVTFTSTASFSESGKVNKGFRAEMEYRTTQTPSNLATLFSAMGNIFVRANGSNLEYGFSTNPSGSTWNDYTKSVTLPSNNVKHIIQLTYLPGADGAASTLQLSVDGVAGETATSAAGELAAVSDSVGNKFGIGYEVNPASGAASRGLAGDVFRARVADSDAPWEILDASQLLHVNFNGTFSGTSYTAASGEQMLGSLVSRSANPSISNSAVTLGGGTAGFDFTPTDFTLGDNEAITRPLVAELRFTPTQTGDNQTLFGAGGNLFLRYESNKLVFGASTKSGDNWTDHKIESAAATGAEHVVSVAYVPNKAGTGAKLVMRVDGGDAQTKDITGLAYLNSSIKGKVGFGNDVHTDALSRGFVGSLSEIRLAETSANFTTNEFKLVYSQVSCDTSGIKEANTFDVEPAECEAALKTKLSKLRPTEGQADYIDWGQIGFLHYGINTYYNQEWGHGNEDPSRINPTGLDTDQWAKSFADGGFKMIMVTVKHHDGFELYDSRYNTEHDWANTAVAKRTGEKDLFRKIVASAKKYGLKVGIYYSPADSYMERKGVWGNNSARVERTIPTLVENDDRAGKVASGKLPTFKYKATDYGAYMLNQLYELLTEYGDISEVWFDGAQGNTAGTEHYDYGVFYEMIRRLQPQAIQANAAYDARWVGNEDGWARQTEWSPQAAYNDGVDKVSLKPGQMAPDGKLGSMSSVLSEIRSGAANQLHWYPAEVDAKNRPGWFYRASQSPASVAEVVKYYEQSTGRNSQYLLNVPPSDTGKLADADAAGLKGLGEELARRYGTDLALGKSATVAASANDTAVAAPKLTDGSKLSSDKAVGNTPTYTIDLGSTVAVDAVKISEDVRNAGQQIESATLQGRVNGTWTNLATMTTVGQQRDLRFTSQNIDAIRLVVNSSRGPVRLSRLEVFHTESEIQTGARAYYIDPTAQTAGDGFTKDKPMTSIEQLHDVTVAPGSVIFVKAGTELTGDFAVFGYGTKDEPITVTTYGESDKATTASFDGMTAGLTLKQALKALGKDDAGWVVADSATAPASRVYVPQDEISVHAQSSQNSGAEAARALDGDSSTSWHSQYSPTTASAPHWVTLDLGKSRENVAYFDYLARIDGNNNGAAKDYEVYVSDDPNDFGAPVASGTLKNVAYTQRIKLTPKNGRYVKFVIKTDYSGSNFGSAAEMNVELLPTAVEEDKVATPQKPTVDDDADTYTIPDIEGVVYKVDGKVLAAGSVVNVGDEDVTVTVTAEPADGYRFPDGVTSPVTYELTFTKKGGEKPPTEVNKDKLHATITKAQAIDRSAYTDESLKVLDDKLAAALKVYDDDKVSQDDVDAAEAALSAAIDALKTKPTTPGGEGEKPGEGEKPGDGNKPGDGKKPGDVIAKTGASTMGVVFAALAMVAGAVVTLEAKRKSNR

SEQ ID NO: 5 - protein, 478 aa, Bifidobacterium longum
  Deposit numbergi|213524647|gb|ACJ53394.1| alpha-1,3/4-fucosidase, putative [Bifidobacterium longum subsp. infantis ATCC 15697]
MNNPADAGINLNYLANVRPSSRQLAWQRMEMYAFLHFGMNTMTDREWGLGHEDPALFNPRNVDVDQWMDALVAGGMAGVILTCKHHDGFCLWPSRLTRHTVASSPWREGKGDLVREVSESARRHGLKFGVYLSPWDRTEESYGKGKAYDDFYVGQLTELLTQYGPIFSVWLDGANGEGKNGKTQYYDWDRYYNVIRSLQPDAVISVCGPDVRWAGNEAGHVRDNEWSVVPRRLRSAELTMEKSQQEDDASFATTVSSQDDDLGSREAVAGYGDNVCWYPAEVDTSIRPGWFYHQSEDDKVMSADQLFDLWLSAVGGNSSLLLNIPPSPEGLLAEPDVQSLKGLGRRVSEFREALASVRCEARTSSASAAAAHLVDGNRDTFWRPDADDAAPAITLTLPQPTTINAIVIEEAIEHGQRIEHLRVTGALPDGTERVLGQAGTVGYRRILRFDDVEVSSVTLHVDGSRLAPMISRAAAVRI

SEQ ID NO: 6 - protein, 378 aa, Lactobacillus casei
  Deposit number gi|190713109|emb|CAQ67115.1| Alpha-L-fucosidase [Lactobacillus casei BL23]
MTKPSAVNIHRTNPPDWFKQADFGIIIHWGPYSVPAYAPVNIPDYGTLIKTKSLGYLFEHQPYAEWYGNSMLLPSSPVAAYHRTHYGNTSYADFAKTFKQTAQNVDVQAWAAAFANAGAKYVVIVTKHHDGFVMFDPHTKNPYEPDYHLNFDFVGELAQAVRAHGMRFGTYYSSLLDWTFPHLPIKDYGSFLLGNDKSQTYKDYVWHQWHELIDRYHPDVLWNDIGYPDDHRLETLFKYYYQQVPEGLVNDRWQQFPDWMRTSWIRPIFNLVAAQVIKRDQHHSNDLSEVKYYDYRTFEYRTDWPQTNRYFEMTRGMDKSFGYNRLSRPEDYITADDIKQLIAELRSKRGRLLLNVGPDMNGQIPSAQLSILQQLSDR

SEQ ID NO: 7 - protein, 414 aa, Lactobacillus casei
  Deposit number gi|190713871|emb|CAQ67877.1| Alpha-L-fucosidase [Lactobacillus casei BL23]
MTEPLPRIQHYEDLGLGLFIHWGLYSQMAVGEWTELIHHRNQHDYEQLIKTFTAAQFDAKKIAHAAKAVGAKYIVLTTKHHEGFFLYDTKGLSDFDVMHAPARRDLIAEFVAACREEDLLPFFYMATYDWHTPLYDDDFPAYLTYLQKSVEVLCRNYGPVGGFWFDGNWNKKDADWHLPELYGMIRHYQPNAIIVNNTGLKNRGQVSDPEIDVVTYERRTPDEIYHGAPNEKYVAGEISITLNQHWGIAANDLNYKSPAEVIETVAHARHIGANILVNIGLTGTGAIPAAAQTYMHLLGRWTAMAAPVLYKGRPVPVTSAHGTRDFVLHTSKHDFLCILDLQVVGNDNVVLGGEGVNPRSFVGIGQPIQRIHWLDNDEVLSFTQDLDKKVLTVDATGYPYGSDWVVRIAQIDYE

SEQ ID NO: 8 - protein, 344 aa, Lactobacillus casei
  Deposit number gi|190713978|emb |CAQ67984.1| Alpha-L-fucosidase [Lactobacillus casei BL23]
MNDNVAWFKQAKYGMMIHWGLYSLLAGEYRGESSSAYAEWIQSKFQIPNAEYGNLATAFNPLYFDAKKIVALAKQCGMQYLVVTTKHHDGFAMYHSKVDAYNVYDATPFHRDIIGELAEACQKAGLKFGLYYSQDLDWHDPNGGGYKSNDVETAGTTWDNSWDFPDEDQKNFDLCFDNKILPQIKEIMSNYGDIATAWFDVPMTLSEAQSQTIYDTVRELQPNCLINSRLGNGKYDFVSLGDNEIPKNKEDMNKTDVDYNEITGFKPSPLGLYETAGTINDSWGFSYHDQNWKTPRTLYRYKQHLNDFGINYLLNVGLDPLGRVPMMAEENLLAAKALEDEANR

(19) United States
(12) Patent Application Publication
(10) Pub. No.: US 2014/0228554 A1
(43) Pub. Date: Aug. 14, 2014

(54) SYNTHESIS OF NEW FUCOSE-CONTAINING CARBOHYDRATE DERIVATIVES

(71) Applicant: Glycom A/S, Kgs. Lyngby (DK)

(72) Inventors: Elise Champion, Toulouse (FR); Gyula Dekany, Sinnamon Park (AU); Markus Hederos, Svedala (SE); Karoly Agoston, Telki (HU)

(73) Assignee: Glycom A/S, Kgs. Lyngby (DK)

(21) Appl. No.: 14/005,796

(22) PCT Filed: Mar. 19, 2012
(86) PCT No.: PCT/IB2012/051314

§ 371 Date: Nov. 5, 2013

(30) Foreign Application Priority Data
Mar. 18, 2011 (GB) 1104611.7
May 13, 2011 (EP) 11166005.6
May 13, 2011 (EP) 11166135.1
May 13, 2011 (EP) 11166137.7

(51) Int. Cl. C12P 19/60; C07H 15/203; C07H 15/18; C07H 17/04

(52) U.S. Cl. CPC C12P 19/60; C07H 17/04; C07H 15/203; C07H 15/18
USPC 536/4.1; 536/18.7; 435/75

(57) ABSTRACT

A method for the synthesis of a fucooligosaccharide glycosides by reacting a fucosyl donor with H-A-R₁ or a salt thereof, wherein A and R₁ are as defined herein, under the catalysis of an enzyme capable of transferring fucose is provided. The fucooligosaccharid glycoside compounds, or derivatives thereof, their use in the manufacture of human milk oligosaccharides, and a method of manufacture of human milk oligosaccharides, are also provided.

### FIELD OF THE INVENTION

The present invention relates to the enzymatic synthesis of fucooligosaccharide glycosides.

### BACKGROUND OF THE INVENTION

Human milk oligosaccharides (HMOs) are of great importance which is directly linked to their unique biological activities such as antibacterial, antiviral, immune system and cognitive development enhancing activities. HMOs are found to act as prebiotics in the human intestinal system helping to develop and maintain the intestinal flora.

Furthermore, they have also proved to be anti-inflammatory substances, and therefore they are attractive components in the nutritional industry for the production of, for example, infant formulas, infant cereals, clinical infant nutritional products, toddler formulas, or as dietary supplements or health functional food for children, adults, elderly or lactating women, both as synthetically composed and naturally occurring compounds and salts thereof. The HMOs are also of interest in the medicinal industry for the production of therapeutics.

In fucose containing HMOs, the fucose residue can be linked to the 2-O-position of D-galactose, the 3-O-position of D-glucose and the 3- or 4-O-position of N-acetylglucosamine via α-glycosidic linkage. The most important fucosylated HMOs are 2′-O-fucosyllactose (Fucα1-2Gal 1-4Glc), 3-O-fucosyllactose (Galβ1-4-[Fucα1-3]Glc), 3′-O-sialyl-3-O-fucosyl-lactose (NeuAcα2-3Galβ1-4-[Fucα1-3]Glc), difucosyllactose (Fucα1-2Galβ1-4-[Fucα1-3]Glc), lacto-N-fucopentaose I (Fucα1-2Galβ1-3GlcNAcβ1-3Galβ1-4Glc), lacto-N-fucopentaose II (Galβ1-3-[Fucα1-4]GlcNAcβ-3Galβ1-4Glc), lacto-N-fucopentaose III (Galβ1-4-[Fucα1-3]GlcNAcβ1-3Galβ1-4Glc), lacto-N-fucopentaose V (Galβ1-3GlcNAcβ1-3Galβ1-4-[Fucα1-3]Glc), lacto-N-difuco-hexaose I (Fucα1-2Galβ1-3-[Fucα1-4]GlcNAcβ1-3Gal 1-4Glc), lacto-N-difuco-hexaose II (Galβ1-3-[Fucα1-4]GlcNAcβ1-3Galβ1-4-[Fucα1-3]Glc), lacto-N-difuco-hexaose III (Galβ1-4-[Fucα1-3]GlcNAcβ1-3Galβ1-4-[Fucα1-3]Glc), F-LST a (NeuAcα2-3Galβ1-3 [Fucα1-4]GlcNAcβ1-3Galβ1-4Glc), F-LST b (Fucα1-2Galβ1-3-[NeuAcα2-6]GlcNAcβ1-3Galβ1-4Glc), F-LST c (NeuAcα2-6Galβ1-4GlcNAcβ1-3Galβ1-4-[Fucα1-3]Glc), fucosyl-lacto-N-(neo)hexaoses, difucosyl-lacto-N-(neo)hexaoses, sialyl-fucosyl-lacto-N-(neo)hexaoses, sialyl-difucosyl-lacto-N-(neo)hexaoses and trifucosyl-lacto-N-(neo)hexaoses [C. Kunz et al. Annu. Rev. Nutr. 20, 699 (2000), T. Urashima et al.: Milk Oligosaccharides, Nova Science Publishers, New York (2011) and references cited therein].

The availability of naturally occurring HMOs is limited. Mature human milk is the natural milk source that contains the highest concentrations of HMOs (12-14 g/l), other milk sources are cow's milk (0.01 g/l), goat's milk and milk from other mammals. The isolation of fucooligosaccharides from human and other mammalian milk is also rather difficult even in milligram quantities due to the presence of a large number of similar oligosaccharides. To date only analytical HPLC methodologies have been developed for the isolation of some fucooligosaccharides from natural sources of HMOs. Their low natural availability and their difficult isolation are important reasons for the development of biotechnological and chemical methodologies for the production of HMOs.

The chemical synthesis of complex fucooligosaccharides requires multistep synthetic pathways utilising protection and deprotection strategies. Stereoselective chemical synthetic processes can become complicated due to the extensive use of protecting groups. These strategies give fucosylated oligosaccharides via stereoselective O-fucosylation of appropriate protected glycosyl acceptors using glycosyl halide, thioglycoside or trichloroacetimidate donor activations. The use of either expensive or toxic chemicals for the fucosylation such as mercury cyanide, mercury bromide, silver carbonate or bromine is one of the reasons that make such methodologies less attractive.

Inefficient stereocontrol and/or moderate yields likewise make(s) the strategies less suitable for further development. Additionally, these strategies are characterized by tedious manipulations and severe purification difficulties.

In the enzymatic production of fucooligosaccharides, fucosyltransferases and fucosidases have been the preferred enzymes used. Although enzymatic fucosylation usually occurs with high regio- and stereoselectivity, these complex enzymatic systems require expensive methodologies for scaled-up production and difficult purification protocols which are likewise a hindrance for further technology developments. These drawbacks seem to be gradually overcome by new achievements in enzyme engineering [see reviews: S. M. Hancock et al. Curr. Opin. Chem. Biol. 10, 509 (2006), R. Kittl et al. Carbohydr. Res. 345, 1272 (2010) and references cited therein]. Recently transfucosidases and fucosynthases as mutant fucosidases with improved fucosylation activity have been developed [G. Osanjo et al. Biochemistry 46, 1022 (2007), J. Wada et al. FEBS Lett. 582, 3739 (2008), B. Cobucci-Ponzano et al. Chem. Biol. 16, 1097 (2009)].

Isolation technologies have never been able to provide large quantities of fucooligosaccharides due to the large number of oligosaccharides present in the pool of natural origin, e.g. in human milk. Additionally, the presence of regioisomers characterized by extremely similar structures has made separation technologies unsuccessful.

Recently, sialooligosaccharide derivatives optionally substituted with fucose have been disclosed (WO 2012/007588).

During the past decades the interest in the preparation and commercialisation of fucosylated HMOs has been increasing steadily. Hence, there has been a need for methodologies which can simplify their preparation and overcome or avoid previous purification problems.

### SUMMARY OF THE INVENTION

In a first aspect, the present invention provides a method for the synthesis of a compound of formula 1 or a salt thereof,

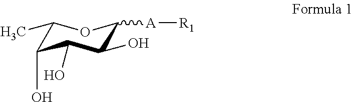

wherein A is a carbohydrate linker which is a lactosyl moiety or consists of a lactosyl moiety and at least one monosaccharide unit selected from the group consisting of: glucose, galactose, N-acetylglucosamine, fucose and N-acetyl neuraminic acid; and wherein R₁ is one of the following anomeric protecting groups:
   a) —OR₂, wherein R₂ is a protecting group removable by catalytic hydrogenolysis,
   b) —SR₃, wherein R₃ is an optionally substituted alkyl, an optionally substituted aryl or an optionally substituted benzyl,
   c) —NH—C(R″)═C(R′)₂, wherein each R′ independently is one of the following electron withdrawing groups: —CN, —COOH, —COO-alkyl, —CO-alkyl, —CONH₂, —CONH-alkyl or —CON(alkyl)₂, or wherein the two R′-groups are linked together and form —CO—(CH₂)₂₋₄—CO— and thus form, together with the carbon atom to which they are attached, a 5-7 membered cycloalkan-1,3-dione, in which dione any of the methylene groups is optionally substituted with 1 or 2 alkyl groups, and R″ is H or alkyl,
      in which a fucosyl donor of formula 2

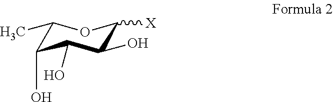

wherein X is selected from the group consisting of: a guanosine diphosphatyl moiety, a lactose moiety, azide, fluoride, optionally substituted phenoxy, optionally substituted pyridinyloxy, optionally substituted 3-oxo-furanyloxy of formula A, optionally substituted 1,3,5-triazinyloxy of formula B, 4-methylumbelliferyloxy-group of formula C, and a group of formula D

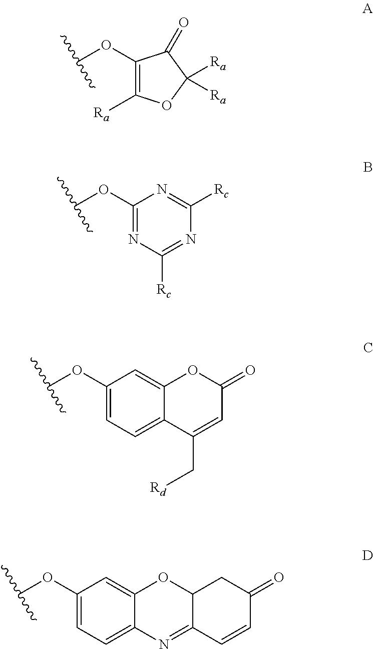

wherein Rₐ is independently H or alkyl, or two vicinal Rₐ groups represent a ═C(R_{b})₂ group, wherein R_{b} is independently H or alkyl, Rₑ is independently selected from the group consisting of alkoxy, amino, alkylamino and dialkylamino, R_{d} is selected from the group consisting of H, alkyl and —C(═O)Rₑ, wherein Rₑ is OH, alkoxy, amino, alkylamino, dialkylamino, hydrazino, alkylhydrazino, dialkylhydrazino or trialkylhydrazino,
   is reacted with an acceptor of formula H-A-R₁ or a salt thereof, wherein A and R₁ are as defined above, under the catalysis of an enzyme capable of transferring fucose.

Preferably, the enzyme is selected from the group consisting of fucosyltransferases and fucosidases and more preferably a fucosidase that is an engineered transfucosidase or engineered fucosynthase. Preferably, the engineered transfucosidase or the engineered fucosynthase stems from Bifidobacterium bifidum, Sulfolobus solfataricus or Thermotoga maritima. More preferably, the fucosidase enzyme is an engineered α-transfucosidase and either the compound of formula 2 is 2′-O-fucosyllactose, or X in formula 2 is selected from the group consisting of phenoxy-, p-nitrophenoxy-, 2,4-dinitrophenoxy-, 2-chloro-4-nitrophenoxy-, 4,6-dimethoxy-1,3,5-triazin-2-yloxy-, 4,6-diethoxy-1,3,5-triazin-2-yloxy-, 2-ethyl-5-methyl-3-oxo-(2H)-furan-4-yloxy-, 5-ethyl-2-methyl-3-oxo-(2H)-furan-4-yloxy- or 2,5-dimethyl-3-oxo-(2H)-furan-4-yloxy-group.

Preferably, the acceptor is a defucosylated human milk oligosaccharide in anomerically protected form. Preferably, A and R₁ are defined as for the preferred features of the second aspect of the invention below.

In a second aspect, the present invention provides a compound of formula 1 or a salt thereof,

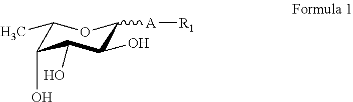

wherein A is a carbohydrate linker which is either a lactosyl moiety or consists of a lactosyl moiety and at least one monosaccharide unit selected from the group consisting of: glucose, galactose, N-acetylglucosamine, fucose and N-acetyl neuraminic acid; and wherein R₁ is one of the following anomeric protecting groups:
   a) —OR₂, wherein R₂ is a protecting group removable by catalytic hydrogenolysis,
   b) —SR₃, wherein R₃ is an optionally substituted alkyl, an optionally substituted aryl or an optionally substituted benzyl,
   c) —NH—C(R″)═C(R′)₂, wherein each R′ independently is one of the following electron withdrawing groups: —CN, —COOH, —COO-alkyl, —CO-alkyl, —CONH₂, —CONH-alkyl or —CON(alkyl)₂, or wherein the two R′-groups are linked together and form —CO—(CH₂)₂₋₄—CO— and thus form, together with the carbon atom to which they are attached, a 5-7 membered cycloalkan-1,3-dione, in which dione any of the methylene groups is optionally substituted with 1 or 2 alkyl groups, and R″ is H or alkyl,
   provided that if R₁ is —OR₂ then linker A does not comprise N-acetyl neuraminic acid.

Preferably, the compound according to the present invention is characterized by formula 1′

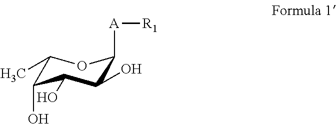

wherein A and R₁ are as defined above.

Preferably, the carbohydrate linker A together with the terminal fucosyl moiety forms a human milk oligosaccharide glycosyl residue. Preferably, the carbohydrate linker A comprises lactosaminyl residue(s) and/or isolactosaminyl residue(s). Preferably, in the carbohydrate linker A the lactosyl moiety is at the reducing end of the linker.

The compounds according to the present invention are preferably selected from the group consisting of β—R₁-glycosides of: 2′-O-fucosyllactose, 3-O-fucosyllactose, 3′-O-sialyl-3-O-fucosyl-lactose, difucosyllactose, lacto-N-fucopentaose I, lacto-N-fucopentaose II, lacto-N-fucopentaose III, lacto-N-fucopentaose V, lacto-N-difuco-hexaose I, lacto-N-difuco-hexaose II, lacto-N-difuco-hexaose III, F-LST a, F-LST b and F-LST c. More preferably, the compounds according to the present invention are selected from the group consisting of β—OR₂- and β-SR₃-glycosides of: 2′-O-fucosyllactose, 3-O-fucosyllactose, difucosyllactose, lacto-N-fucopentaose I, lacto-N-fucopentaose II, lacto-N-fucopentaose III, lacto-N-fucopentaose V, F-LST a, F-LST b and F-LST c. Preferably, R₂ is a benzyl or 2-naphthylmethyl group, each of which is optionally substituted with at least one group selected from the group consisting of phenyl, alkyl or halogen, or R₃ is phenyl or benzyl.

In a third aspect, the present invention provides the use of a compound of formula 1′ or a salt thereof of the second aspect in the synthesis of fucosylated human milk oligosaccharides and salts thereof.

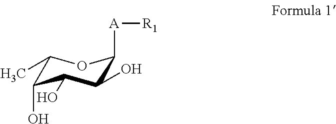

wherein A and R₁ are as defined above and preferably wherein A together with the terminal fucosyl moiety is a human milk oligosaccharide glycosyl residue,
   the synthesis comprising the step of removing the anomeric protecting group R₁.

Preferably, A and R₁ are defined as for the preferred features of the second aspect of the invention above.

In a fourth aspect, the present invention provides a method of manufacture of a human milk oligosaccharide or a salt thereof, comprising removing the anomeric protecting group R₁ from a compound of formula 1′ or a salt thereof

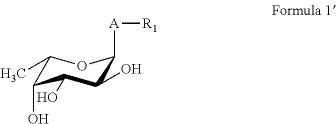

wherein A is a carbohydrate linker which is a lactosyl moiety or which consists of a lactosyl moiety and at least one monosaccharide unit selected from the group consisting of: glucose, galactose, N-acetylglucosamine, fucose and N-acetyl neuraminic acid; and wherein R₁ is one of the following anomeric protecting groups:
   a) —OR₂, wherein R₂ is a protecting group removable by catalytic hydrogenolysis,
   b) —SR₃, wherein R₃ is an optionally substituted alkyl, an optionally substituted aryl or an optionally substituted benzyl,
   c) —NH—C(R″)═C(R′)₂, wherein each R′ independently is one of the following electron withdrawing groups: —CN, —COOH, —COO-alkyl, —CO-alkyl, —CONH₂, —CONH-alkyl or —CON(alkyl)₂, or wherein the two R′-groups are linked together and form —CO—(CH₂)₂₋₄—CO— and thus form, together with the carbon atom to which they are attached, a 5-7 membered cycloalkan-1,3-dione, in which dione any of the methylene groups is optionally substituted with 1 or 2 alkyl groups, and R″ is H or alkyl
   provided that if R₁ is —OR₂ then linker A does not comprise N-acetyl neuraminic acid.

Preferably, the compound of formula 1 or the salt thereof is formed by the method of the first aspect of the invention. Preferably, the method comprises the use of the compound of formula 2A of the sixth aspect as the fucosyl donor in the formation of the compound of formula 1 or the salt thereof according to the method of the first aspect of the invention, and more preferably the method comprises forming the compound of formula 2A according to the method of the seventh aspect of the invention.

Preferably, A and R₁ are defined as for the preferred aspects of the second aspect of the invention above.

In a fifth aspect, the present invention provides a method of manufacture of a fucosylated oligosaccharide or a salt thereof, comprising the steps of:

synthesis of a compound of formula 1 or a salt thereof in accordance with the first aspect of the invention, and
removing the anomeric protecting group R₁ from the compound of formula 1 or the salt thereof.

Preferably, the method comprises the use of the compound of formula 2A of the sixth aspect as the fucosyl donor in the formation of the compound of formula 1 or the salt thereof according to the method of the first aspect of the invention, and more preferably the method comprises forming the compound of formula 2A according to the method of the seventh aspect of the invention.

Preferably, A and R₁ are defined as for the preferred aspects of the second aspect of the invention above.

In a sixth aspect, the present invention provides a compound of formula 2A

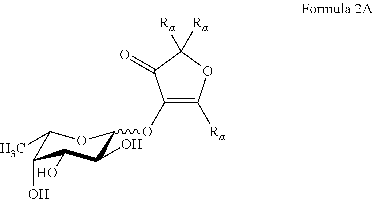

wherein Rₐ is independently H or alkyl, or two vicinal Rₐ groups represent a ═C(R_{b})₂ group, wherein R_{b} is independently H or alkyl, and preferably wherein Rₐ is independently H, methyl or ethyl.

Preferably, the compound of formula 2A is used as the fucosyl donor in the first, third, fourth or fifth aspects of the invention.

In a seventh aspect, the present invention provides a method of synthesis of the compound of the sixth aspect of the invention, comprising the steps of:

a) coupling a fucosyl derivative of formula 3

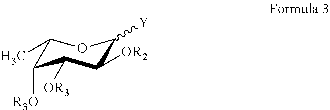

wherein R₂ and R₃ are, independently, a group removable by hydrogenolysis or acyl and Y is a halogen, —OC(═NH)CCl₃, —O-pentenyl, —OAc, —OBz or —SR₄, in which R₄ is alkyl or optionally substituted phenyl with the compound of formula 4

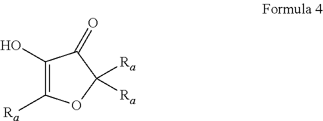

wherein Rₐ is defined as above, and removing the R₂ and R₃ protecting groups.

Preferably, a compound of formula 3 wherein R₂ and R₃ are identical and are each benzyl, 4-methoxybenzyl or 4-methylbenzyl, and Y is trichloroacetimidate is coupled with 2,5-dimethyl-4-hydroxy-3-oxo-(2H)-furan followed by catalytic hydrogenolysis.

In a eighth aspect, the present invention provides a compound made according to the method of the first aspect of the invention.

In a ninth aspect, the present invention provides compounds, methods and uses substantially as described herein.

All features described in connection with any aspect of the invention can be used with any other aspect of the invention.

### DETAILED DESCRIPTION OF THE INVENTION

The present invention provides fucooligosaccharides and salts thereof protected in the anomeric position and a method for their manufacture.

Whatever route is taken to manufacture an oligosaccharide, the final target product as an unprotected oligosaccharide is soluble only in water which presents challenges for the later steps of the manufacturing process. Organic solvents commonly used in the manufacturing process are not suitable in the reactions of the later stages of the process.

The present invention is based upon the utilisation of water soluble 1-O, 1-S or 1-N-protected oligosaccharide intermediates and salts thereof in an enzymatic fucosylation reaction, wherein a selected protecting group can then be removed in simple, clean and nearly quantitative reaction giving rise to fucosylated glycans. Preferably, the anomeric protecting group should also provide the oligosaccharide intermediate with physical and chemical properties assisting powerful purification processes. For example, the introduction of a hydrophobic moiety enables the derivatives to be soluble in organic protic solvents like alcohols while their water solubility also remains. This opens the possibility of using mobile phases having a wide range of water/alcohol proportions which can be applied in separation/purification techniques such as size exclusion or reverse phase chromatography. Moreover, with careful design of substituents on the group introduced, crystalline compounds can in some cases be realized, which allows the development of powerful manufacturing procedures using crystallisation alone for product purifications. Furthermore, the aromatic 1-O-protecting group can be removed by catalytic hydrogenolysis in the last step under mild and delicate conditions that prevent by-product formation, which is undoubtedly an advantage. It is possible for the catalytic reduction to take place in aqueous solution.

### GENERAL TERMS

In this invention, the term “carbohydrate linker which is a lactosyl moiety or which consists of a lactosyl moiety and at least one monosaccharide unit selected from the group consisting of glucose, galactose, N-acetylglucosamine, fucose and N-acetyl neuraminic acid” means an unprotected lactose residue which is attached to the R₁-group by the C-1 anomeric carbon atom. The lactose portion can be the part of an oligosaccharide having monosaccharide units selected from the group consisting of glucose, galactose, N-acetylglucosamine, fucose and N-acetyl neuraminic acid and representing a linear or branched structure. The monosaccharides in the carbohydrate linker A have unprotected and unsubstituted OH groups, except for those OH groups involved in interglycosidic linkages and the anomeric OH of the reducing end. The terminal fucosyl moiety is linked to one of the hydroxyl groups of the above specified lactose or lactose containing oligosaccharide residue.

Herein, the term “protecting group removable by catalytic hydrogenolysis” means a group that has a C—O bond with one of the oxygens, preferably with the 1-oxygen of the compound of formula 1 and that is cleaved by hydrogen in the presence of catalytic amounts of palladium, Raney nickel or another metal catalyst known for use in hydrogenolysis, resulting in demasking the parent hydroxy group. Such protecting groups are well known and are discussed in P. G. M. Wuts and T. W. Greene: Protective Groups in Organic Synthesis John Wiley & Sons, 2007. Suitable protecting groups include benzyl, diphenylmethyl(benzhydryl), 1-naphthylmethyl, 2-naphthylmethyl or triphenylmethyl(trityl) groups, each of which can be optionally substituted by one or more groups selected from: alkyl, alkoxy, phenyl, amino, acylamino, alkylamino, dialkylamino, nitro, carboxyl, alkoxycarbonyl, carbamoyl, N-alkylcarbamoyl, N,N-dialkylcarbamoyl, azido, halogenalkyl or halogen. Preferably, such substitution, if present, is on the aromatic ring(s). Particularly preferred protecting groups are benzyl or 2-naphthylmethyl groups optionally substituted with one or more groups selected from phenyl, alkyl or halogen. More preferably, the protecting group is selected from unsubstituted benzyl, unsubstituted 2-naphthylmethyl, 4-chlorobenzyl, 3-phenylbenzyl and 4-methylbenzyl. These particularly preferred and more preferable protecting groups have the advantage that the by-products of the hydrogenolysis are exclusively toluene, 2-methylnaphthalene, or substituted toluene or 2-methylnaphthalene derivatives, respectively. Such by-products can easily be removed even in multi ton scales from water soluble oligosaccharide products via evaporation and/or extraction processes.

Also herein, “alkyl” means a linear or branched chain saturated hydrocarbon group with 1-6 carbon atoms, such as methyl, ethyl, n-propyl, i-propyl, n-butyl, i-butyl, s-butyl, t-butyl, n-hexyl, etc.

Further herein, “removing the anomeric protecting group R₁” means converting the R₁ group into a hydroxyl group.

Also, “aryl” means a homoaromatic group such as phenyl or naphthyl.

Also, “acyl” means an R—C(═O)-group, wherein R can be H, alkyl (see above) or aryl (see above), such as formyl, acetyl, propionyl, butyryl, pivaloyl, benzoyl, etc. The alkyl or aryl residue can either be unsubstituted or can be substituted with one or more groups selected from alkyl (only for aryl residues), halogen, nitro, aryl, alkoxy, amino, alkylamino, dialkylamino, carboxyl, alkoxycarbonyl, carbamoyl, N-alkylcarbamoyl, N,N-dialkylcarbamoyl, azido, halogenalkyl or hydroxyalkyl, giving rise to acyl groups such as chloroacetyl, trichloroacetyl, 4-chlorobenzoyl, 4-nitrobenzoyl, 4-phenylbenzoyl, 4-benzamidobenzoyl, 4-(phenylcarbamoyl)-benzoyl, glycolyl, acetoacetyl, etc.

“Alkyloxy” or “alkoxy” means an alkyl group (see above) attached to the parent molecular moiety through an oxygen atom, such as methoxy, ethoxy, t-butoxy, etc.

“Halogen” means fluoro, chloro, bromo or iodo.

“Amino” means a —NH₂ group.

“Alkylamino” means an alkyl group (see above) attached to the parent molecular moiety through an —NH-group, such as methylamino, ethylamino, etc.

“Dialkylamino” means two alkyl groups (see above), either identical or different ones, attached to the parent molecular moiety through a nitrogen atom, such as dimethylamino, diethylamino, etc.

“Acylamino” means an acyl group (see above) attached to the parent molecular moiety through an —NH-group, such as acetylamino (acetamido), benzoylamino (benzamido), etc.

“Carboxyl” means a —COOH group.

“Alkyloxycarbonyl” means an alkyloxy group (see above) attached to the parent molecular moiety through a —C(═O)-group, such as methoxycarbonyl, t-butoxycarbonyl, etc.

“Carbamoyl” means an H₂N—C(═O)-group.

“N-Alkylcarbamoyl” means an alkyl group (see above) attached to the parent molecular moiety through a —HN—C(═O)-group, such as N-methylcarbamoyl, etc.

“N,N-Dialkylcarbamoyl” means two alkyl groups (see above), either identical or different ones, attached to the parent molecular moiety through a >N—C(═O)-group, such as N,N-methylcarbamoyl, etc.

Fucosylated Oligosaccharides

The present invention provides fucosylated oligosaccharides of formula 1 and salts thereof,

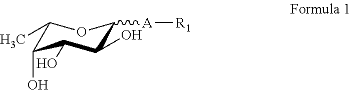

wherein A is a carbohydrate linker which is a lactosyl moiety or which consists of a lactosyl moiety and one or more monosaccharide units selected from the group consisting of: glucose, galactose, N-acetylglucosamine, fucose and N-acetyl neuraminic acid; and wherein R₁ is selected from the group consisting of
   a) —OR₂, which R₂ is a group removable by catalytic hydrogenolysis,
   b) —SR₃, which R₃ is selected from optionally substituted alkyl, optionally substituted aryl and optionally substituted benzyl and
   c) —NH—C(R″)═C(R′)₂, wherein each R′ independently of each other is an electron withdrawing group selected from the group consisting of —CN, —COOH, —COO-alkyl, —CO-alkyl, —CONH₂, —CONH-alkyl and —CON(alkyl)₂, or wherein the two R′-groups are linked together and are —CO—(CH₂)₂₋₄—CO— and thus form, together with the carbon atom to which they are attached, a 5-7 membered cycloalkan-1,3-dione, in which dione any of the methylene groups is optionally substituted with 1 or 2 alkyl groups, and R″ is H or alkyl,
      provided that if R₁ is —OR₂ then linker A does not comprise N-acetyl neuraminic acid.

An oligosaccharide of formula 1 of this invention that contains at least one sialyl residue can be in salt form, which means an associated ion pair consisting of the negatively charged acid residue of the sialylated oligosaccharide and one or more cations in any stoichiometric proportion. The cation(s) can be atoms or molecules with a positive charge and can be inorganic as well as organic. Preferred inorganic cations are the ammonium ion and the alkali metal, alkaline earth metal and transition metal ions, more preferably Na⁺, K⁺, Ca²⁺, Mg²⁺, Ba²⁺, Fe²⁺, Zn²⁺, Mn²⁺ and Cu²⁺, most preferably K⁺, Ca²⁺, Mg²⁺, Ba²⁺, Fe²⁺ and Zn²⁺. Basic organic compounds in positively charged form can be organic cations. Preferred positively charged organic compounds are diethyl amine, triethyl amine, diisopropyl ethyl amine, ethanolamine, diethanolamine, triethanolamine, imidazole, piperidine, piperazine, morpholine, benzyl amine, ethylene diamine, meglumin, pyrrolidine, choline, tris-(hydroxymethyl)-methyl amine, N-(2-hydroxyethyl)-pyrrolidine, N-(2-hydroxyethyl)-piperidine, N-(2-hydroxyethyl)-piperazine, N-(2-hydroxyethyl)-morpholine, L-arginine, L-lysine, oligopeptides having an L-arginine or L-lysine unit and oligopeptides having a free N-terminal amino group, all in protonated form. Such salt formations can be used to modify characteristics of an oligosaccharide of formula 1 as a whole, such as stability, compatibility with excipients, solubility and ability to form crystals.

A preferred embodiment of the invention relates to a compound of formula 1′

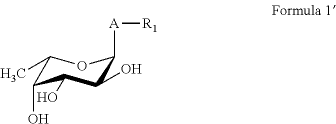

wherein A and R₁ are as defined above.

In a more preferred embodiment, linker A with the terminal fucosyl moiety is a human milk oligosaccharide glycosyl residue. In other words, compounds of formula 1′ encompass fucose containing human milk oligosaccharide R₁-glycosides.

In another more preferred embodiment, linker A comprises a lactosaminyl and/or isolactosaminyl residue(s). Preferably, the lactosaminyl or isolactosaminyl residue is attached to the 3′-OH group of the lactosyl portion.

In yet another more preferred embodiment, R₂ is benzyl or 2-naphthylmethyl groups optionally substituted with at least one group selected from the group consisting of phenyl, alkyl and halogen, and R₃ is phenyl or benzyl.

In an especially preferred embodiment, a compound of formula 1′ is selected from the group consisting of β-R₁-glycosides of 2′-O-fucosyllactose, 3-O-fucosyllactose, 3′-O-sialyl-3-O-fucosyl-lactose, difucosyllactose, lacto-N-fucopentaose I, lacto-N-fucopentaose II, lacto-N-fucopentaose III, lacto-N-fucopentaose V, lacto-N-difuco-hexaose I, lacto-N-difuco-hexaose II, lacto-N-difuco-hexaose III, F-LST a, F-LST b and F-LST c, preferably β-OR₂— and β-SR₃-glycosides of 2′-O-fucosyllactose, 3-O-fucosyllactose, difucosyllactose, lacto-N-fucopentaose I, lacto-N-fucopentaose II, lacto-N-fucopentaose III, lacto-N-fucopentaose V, F-LST a, F-LST b and F-LST c.

An advantage of providing compounds of formula 1 is that it allows a more simple purification of the anomerically protected fucosylated oligosaccharide glycosides compared to the unglycosylated fucooligosaccharides. Due to the different polarities of the reaction compounds, isolation of the compounds of formula 1 by reverse phase or size exclusion chromatography is now possible. In the case of reverse phase chromatography when water is used, compounds of formula 1 migrate much more slowly than the very polar compounds also present in the reaction mixture, and thus, the polar compounds can be eluted smoothly. Compounds of formula 1 can then be washed from the column with e.g. alcohol.

Fucosylation/Transfucosylation Reaction

The present invention also provides a process for synthesizing fucooligosaccharides of formula 1 and salts thereof,

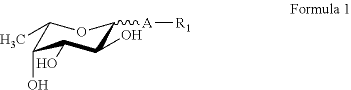

wherein A is a carbohydrate linker which is a lactosyl moiety or which consists of a lactosyl moiety and one or more monosaccharide units selected from the group consisting of glucose, galactose, N-acetylglucosamine, fucose and N-acetyl neuraminic acid; and wherein R₁ is selected from the group consisting of
   a) —OR₂, which R₂ is a group removable by catalytic hydrogenolysis,
   b) —SR₃, which R₃ is selected from optionally substituted alkyl, optionally substituted aryl and optionally substituted benzyl and
   c) —NH—C(R″)═C(R′)₂, wherein each R′ independently of each other is an electron withdrawing group selected from the group consisting of —CN, —COOH, —COO-alkyl, —CO-alkyl, —CONH₂, —CONH-alkyl and —CON(alkyl)₂, or wherein the two R′-groups are linked together and are —CO—(CH₂)₂₋₄—CO— and thus form, together with the carbon atom to which they are attached, a 5-7 membered cycloalkan-1,3-dione, in which dione any of the methylene groups is optionally substituted with 1 or 2 alkyl groups, and R″ is H or alkyl, characterized in that a fucosyl donor of formula 2

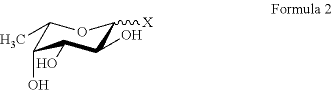

wherein X is selected from the group consisting of guanosine diphosphatyl moiety, a lactose moiety, azide, fluoride, optionally substituted phenoxy-, optionally substituted pyridinyloxy-, optionally substituted 3-oxo-furanyloxy of formula A, optionally substituted 1,3,5-triazinyloxy of formula B, 4-methylumbelliferyloxy-group of formula C and a group of formula D

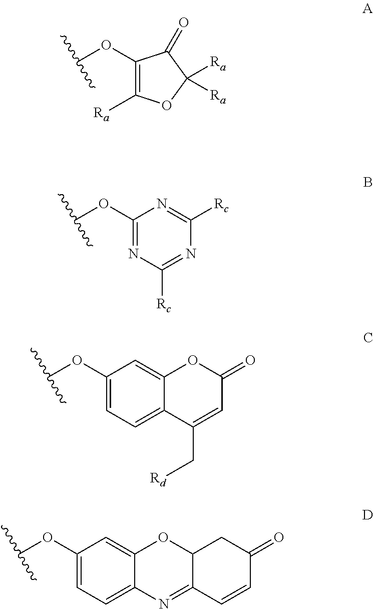

wherein Rₐ is independently H or alkyl, or two vicinal Rₐ groups represent a ═C(R_{b})₂ group, wherein R_{b} is independently H or alkyl, Rₑ is independently selected from the group consisting of alkoxy, amino, alkylamino and dialkylamino, R_{d} is selected from the group consisting of H, alkyl and —C(═O)Rₑ, wherein Rₑ is OH, alkoxy, amino, alkylamino, dialkylamino, hydrazino, alkylhydrazino, dialkylhydrazino or trialkylhydrazino,
   is reacted with an acceptor of formula H-A-R₁
wherein A and R₁ are as defined above,
   under the catalysis of an enzyme capable of transferring fucose. This process is depicted in Scheme 1.

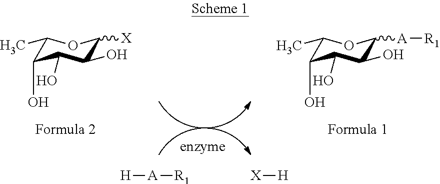

This transfucosylation reaction can be carried out in a conventional manner at a pH of about 4-9, preferably at a temperature of from about 10 to 50° C., preferably from about 30° C. to 40° C., except for thermophilic enzymes wherein the incubation is performed at a temperature of from 50 to 80° C., preferably from 60 to 70° C. In this regard, incubation of the fucosyl donor of formula 2 with the acceptor of formula H-A-R₁ preferably occurs with a concentration of the enzyme of 10 mU/l to 100 U/l, wherein the activity capable of forming 1 μmol of the compound of formula 1 starting from a defined amount of the donor of formula 2 is defined as 1 unit (U). The incubation suitably can be carried out in an aqueous reaction medium, preferably containing a buffer such as a phosphate, carbonate, acetate, borate, citrate or tris buffer, or a combination thereof. A water soluble organic solvent, preferably a C₁-C₄ alcohol, DMF or DMSO, can also be added to the reaction mixture to accelerate the reaction. 0.1 to 50 g/l of a surfactant can also be added to accelerate the reaction. Examples include nonionic surfactants such as polyoxyethylene octadecylamine (e.g., Nymeen S-215, manufactured by Nippon Oil & Fats), cationic surfactants, such as cetyltrimethylammonium bromide and alkyldimethyl benzylammoniumchloride (e.g., Cation F2-40E, manufactured by Nippon Oil & Fats) and anionic surfactants such as lauroyl sarcosinate; tertiary amines such as alkyldimethylamine (e.g., Tertiary Amine FB, manufactured by Nippon Oil & Fats). 0.1 to 50 ml/l of an organic solvent, such as xylene, toluene, a fatty acid alcohol, acetone and ethyl acetate, and 0.1 to 5 g/l of an inorganic salt, such as MnCl₂ or MgCl₂, can also be added to the reaction mixture.

Enzymes Capable of Transferring Fucose in the Fucosylation/Transfucosylation Reaction

In biological systems, fucosyltransferases and fucosidases are able to carry out fucosylation to yield fucooligosaccharides.

Fucosyltransferase enzymes (classified in EC 2.4.1) transfer L-fucose from a fucosyl nucleotide to the acceptor with high degree of regio- and stereochemical control. Various fucosyltransferases can be found in mammals, in which they are mainly located in the Golgi apparatus. The α-1-2 fucosyltransferase transfers fucose to the 2-O-position of galactose. GlcNAc in the 4-O-position can be fucosylated by means of α-1-4 fucosyltransferase, whereas α-1-3 fucosyltransferase catalyzes the transfer of fucose to the 3-O-position of GlcNAc as well as of glucose.

Fucosidases (classified in EC 3.2.1.38 and 3.2.1.51) are widespread in living organisms such as mammals, plants, fungi and bacteria. These enzymes belong to the families 29, and 95 of the glycoside hydrolases (GH29, GH35 and GH95) as defined by the CAZY nomenclature (http://www.cazy.org; B. L. Cantarel et al. Nucleic Acids Res. 37, D233 (2009)). Fucosidases from GH29 are retaining enzymes (3D structure: (3/a)s) whereas fucosidases from GH95 are inverting enzymes (3D structure: (α/α)₆). The substrate specificity of the GH29 family is broad whereas that of the GH95 family is strict to α1,2-linked fucosyl residues. The GH29 family seems to be divided into two subfamilies. One subfamily typically has strict specificity towards α1,3- and α1,4-fucosidic linkages. The members of a further subfamily have broader specificity, covering all α-fucosyl linkages. Fucosidases generally hydrolyse the terminal fucosyl residue from glycans. However these enzymes are able to act as catalyst for fucosylation reaction due to their transfucosylation activity under kinetically controlled conditions.

The utility of glycosidases, including fucosidases, has benefited from various engineering techniques.

In the technique of “rational engineering”, novel altered enzymes (mutants) are created by point mutation. The mutation generally affects the active site of the enzyme.

Replacement of a catalytic nucleophilic residue with a non-nucleophilic residue results in the formation of an inactive mutant or an altered enzyme with reduced transglycosylation activity due the lack of appropriate environment for the formation of the reactive host-guest complex for transglycosylation. However, in the presence of a more active fucosyl donor than the natural one, the mutated enzyme is able to transfer efficiently the fucose residue to a suitable acceptor. Such a mutant glycosidase is termed glycosynthase. Rational engineering of enzymes generally requires reliance on the static 3D protein structure. By means of rational engineering, an α-1,2-L-fucosynthase from Bifidobacterium bifidum and efficient α-L-fucosynthases from Sulfolobus solfataricus and Thermotoga maritima with acceptor dependent regioselectivity have recently been developed and provided [J. Wada et al. FEBS Lett. 582, 3739 (2008), B. Cobucci-Ponzano et al. Chem. Biol. 16, 1097 (2009)]. These altered enzymes are devoid of product hydrolysis activity.

A second technique of “directed evolution” involves random mutagenesis of a selected natural glycosidase enzyme to create a library of enzyme variants, each of which is altered in a single position or in multiple positions. The variants can be inserted into suitable microorganisms such as E. coli or S. cerevisiae for producing recombinant variants with slightly altered properties. Clones expressing improved enzyme variants are then identified with a fast and reliable screening method, selected and brought into a next round of mutation process. The recursive cycles of mutation, recombination and selection are continued until mutant(s) with the desired activity and/or specificity is/are evolved. An α-L-fucosidase from Thermotoga maritima has recently been converted into an efficient α-L-transfucosidase by directed evolution [G. Osanjo et al. Biochemistry 46, 1022 (2007)]. The cited article describes the cloning, mutation, screening, recombination and protein purification steps in detail.

It is envisaged that transfucosidase and/or fucosynthase enzyme mutants retaining transfucosidase activity and having a sequence similarity/homology to the sequence of the known and published enzyme sequences, such as that of the α-L-transfucosidase of G. Osanjo et al, of at least 70%, such as 75%, preferably 80%, such as 85% can be used in the present invention. Preferably, the sequence similarity is at least 90%, more preferably 95%, 97%, 98% or most preferably 99%.

Engineered transfucosidases and fucosynthases possess a broader donor and acceptor specificity than the wild types of fucosidases and fucosyltransferases and so can be used in a particularly wide variety of reactions. The engineered enzymes are, therefore, more advantageous for industrial use.

Accordingly, a preferred fucosidase enzyme for use in the transfucosylation process of this invention is an engineered fucosidase enzyme, more preferably an α-L-transfucosidase evolved by a directed evolution process from a naturally occurring α-L-fucosidase. Preferably the from the α-L-fucosidase comes from a naturally occurring, thermostable α-L-fucosidase from Thermotoga maritima that is subjected to a directed evolution process with at least one, preferably at least two, more preferably at least three, most preferably at least four, mutation-recombination sequences. Another preferred enzyme for use in the process of this invention is an α-L-fucosynthase evolved by rational engineering methodology from a wild-type α-L-fucosidase. Preferably the wild type α-L-fucosidase is taken from the species Bifidobacterium bifidum, Sulfolobus solfataricus or Thermotoga maritima, and is converted to an α-L-fucosynthase by point mutation.

More preferably, the enzyme having a fucosidase and/or trans-fucosidase activity may be selected from α-L-fucosidases derived from Thermotoga maritima MSB8, Sulfolobus solfataricus P2, Bifidobacterium bifidum JCM 1254, Bifidobacterium bifidum JCM 1254, Bifidobacterium longum subsp. infantis ATCC 15697, Bifidobacterium longum subsp. infantis ATCC 15697, Bifidobacterium longum subsp. Infantis JCM 1222, Bifidobacterium bifidum PRL2010, Bifidobacterium bifidum S17, Bifidobacterium longum subsp longum JDM 301, Bifidobacterium dentium Bdl, or Lactobacillus casei BL23, etc.

Even more preferably the enzyme having a fucosidase and/or trans-fucosidase activity may be selected from following α-L-fucosidases as defined according to the following deposit numbers gi|4980806 (Thermotoga maritima MSB8, SEQ ID NO: 1), gi|3816464 (Sulfolobus solfataricus P2, SEQ ID NO: 2), gi|34451973 (Bifidobacterium bifidum JCM 1254, SEQ ID NO: 3), gi|242345155 (Bifidobacterium bifidum, JCM 1254, SEQ ID NO: 4), gi|213524647 (Bifidobacterium longum subsp. infantis, ATCC 15697, SEQ ID NO: 5), gi|213522629 (Bifidobacterium longum subsp. infantis ATCC 15697), gi|213522799 (Bifidobacterium longum subsp. infantis ATCC 15697), gi|213524646 (Bifidobacterium longum subsp. infantis ATCC 15697), gi|320457227 (Bifidobacterium longum subsp. infantis JCM 1222), gi|320457408 (Bifidobacterium longum subsp. infantis JCM 1222), gi|320459369 (Bifidobacterium longum subsp. infantis JCM 1222), gi|320459368 (Bifidobacterium longum subsp. infantis JCM 1222), gi|310867039 (Bifidobacterium bifidum PRL2010), gi|310865953 (Bifidobacterium bifidum PRL2010), gi|309250672 (Bifidobacterium bifidum S17), gi|309251774 (Bifidobacterium bifidum S17), gi|296182927 (Bifidobacterium longum subsp longum JDM 301), gi|296182928 (Bifidobacterium longum subsp longum JDM 301), gi|283103603 (Bifidobacterium dentium Bdl), gi|190713109 (Lactobacillus casei BL23, SEQ ID NO: 6), gi|190713871 (Lactobacillus casei BL23, SEQ ID NO: 7), gi|190713978 (Lactobacillus casei BL23, SEQ ID NO: 8), etc., or a sequence exhibiting a sequence identity with one of the above mentioned enzyme sequences having a fucosidase and/or trans-fucosidase activity of at least 70%, more preferably at least 80%, equally more preferably at least 85%, even more preferably at least 90% and most preferably at least 95% or even 97%, 98% or 99% as compared to the entire wild type sequence on amino acid level.

Particularly preferred α-L-fucosidases with fucosidase/trans-fucosidase activity are listed in the following Table 1:

TABLE 1
Preferred α-L-fucosidasess

| GI number in | | SEQ |
|---|---|---|
| GenBank | | ID |
| Database | Organisms | NO: |
| gi\|4980806 | Thermotoga maritima MSB8 | 1 |
| gi\|13816464 | Sulfolobus solfataricus P2 | 2 |
| gi\|34451973 | Bifidobacterium bifidum JCM 1254 | 3 |
| gi\|242345155 | Bifidobacterium bifidum JCM 1254 | 4 |
| gi\|213524647 | Bifidobacterium longum subsp. infantis | 5 |
| | ATCC 15697 | |
| gi\|213522629 | Bifidobacterium longum subsp. infantis | — |
| | ATCC 15697 | |
| gi\|213522799 | Bifidobacterium longum subsp. infantis | — |
| | ATCC 15697 | |
| gi\|213524646 | Bifidobacterium longum subsp. Infantis | — |
| | ATCC 15697 | |
| gi\|320457227 | Bifidobacterium longum subsp. infantis JCM 1222 | — |
| gi\|320457408 | Bifidobacterium longum subsp. infantis JCM 1222 | — |
| gi\|320459369 | Bifidobacterium longum subsp. infantis JCM 1222 | — |
| gi\|320459368 | Bifidobacterium longum subsp. infantis JCM 1222 | — |
| gi\|310867039 | Bifidobacterium bifidum PRL2010 | — |
| gi\|310865953 | Bifidobacterium bifidum PRL2010 | — |
| gi\|309250672 | Bifidobacterium bifidum S17 | — |
| gi\|309251774 | Bifidobacterium bifidum S17 | — |
| gi\|296182927 | Bifidobacterium longum subsp longum JDM 301 | — |
| gi\|296182928 | Bifidobacterium longum subsp longum JDM 301 | — |
| gi\|283103603 | Bifidobacterium dentium Bd1 | — |
| gi\|190713109 | Lactobacillus casei BL23 | 6 |
| gi\|190713871 | Lactobacillus casei BL23 | 7 |
| gi\|190713978 | Lactobacillus casei BL23 | 8 |

Donors for the Fucosylation/Transfucosylation Reaction

A compound of formula 2

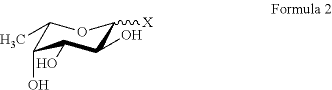

wherein X is selected from the group consisting of guanosine diphosphatyl moiety, a lactose moiety, azide, fluoride, optionally substituted phenoxy-, optionally substituted pyridinyloxy-, optionally substituted 3-oxo-furanyloxy of formula A, optionally substituted 1,3,5-triazinyloxy of formula B, 4-methylumbelliferyloxy-group of formula C and a group of formula D

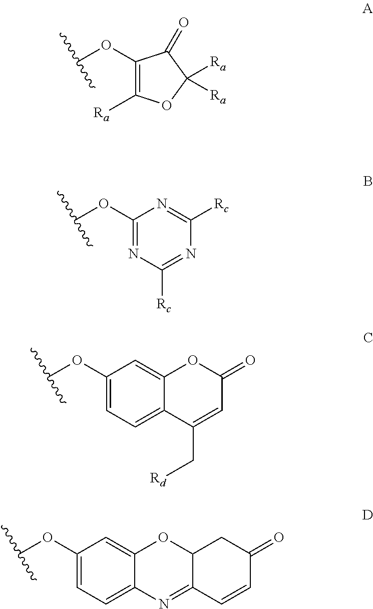

wherein Rₐ is independently H or alkyl, or two vicinal Rₐ groups represent a ═C(R_{b})₂ group, wherein R_{b} is independently H or alkyl, Rₑ is independently selected from the group consisting of alkoxy, amino, alkylamino and dialkylamino, R_{d} is selected from the group consisting of H, alkyl and —C(═O)Rₑ, wherein Rₑ is OH, alkoxy, amino, alkylamino, dialkylamino, hydrazino, alkylhydrazino, dialkylhydrazino or trialkylhydrazino,
   can act as the fucosyl donor in a fucosylation/transfucosylation reaction of this invention.

In a preferred embodiment, a compound of formula 2 is 2′-O-fucosyllactose or wherein X is selected from the group consisting of fluoride, phenoxy-, p-nitrophenoxy-, 2,4-dinitrophenoxy-, 2-chloro-4-nitrophenoxy-, 4,6-dimethoxy-1,3,5-triazin-2-yloxy-, 4,6-diethoxy-1,3,5-triazin-2-yloxy-, 2-ethyl-5-methyl-3-oxo-(2H)-furan-4-yloxy-, 5-ethyl-2-methyl-3-oxo-(2H)-furan-4-yloxy- and 2,5-dimethyl-3-oxo-(2H)-furan-4-yloxy-group is used as the fucosyl donor when an engineered transfucosidase enzyme is employed in the fucosylation reaction. In another preferred embodiment, when the transfucosylation is carried out in the presence of a fucosynthase catalyst, a compound of formula 2 wherein X is fluoride or azide is the donor of choice. In a further preferred embodiment a compound of formula 2 is GDP-fucose when the fucosylation is mediated by a fucosyl transferase enzyme.

Especially preferred fucosyl donors are characterized by formula 2A

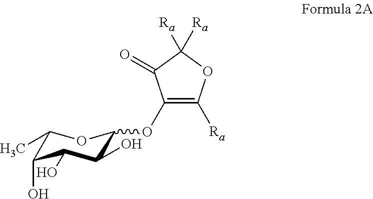

wherein Rₐ is independently H or alkyl, or two vicinal Rₐ groups represent a ═C(R_{b})₂ group, wherein R_{b} is independently H or alkyl, more preferably Rₐ is independently H, methyl or ethyl.

Fucosyl donors of formula 2A are especially advantageous donors in fucosylation/transfucosylation reactions because their water solubility is high, the leaving group after fucosylation can be detected easily by UV detection, and this leaving group, being a natural aroma of fruits, causes no regulatory obstacles when using in the food industry.

Compounds of formula 2A can be synthesized in the reaction comprising the steps of:
a) coupling a fucosyl derivative of formula 3

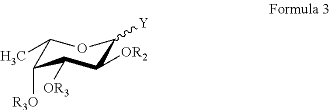

wherein R₂ and R₃ are, independently, a group removable by hydrogenolysis or acyl and Y is a halogen, —OC(═NH)CCl₃, —O-pentenyl, —OAc, —OBz or —SR₄, in which R₄ is alkyl or optionally substituted phenyl
   with the compound of formula 4

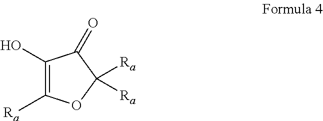

wherein Rₐ is defined as above, and
b) removing the R₂ and R₃ protecting groups.

Reaction step a) can be carried out in a conventional manner in the presence of an activator in an aprotic solvent or mixture of aprotic solvents. The glycosylation reaction is generally promoted by heavy metal ions and Lewis acids. A glycosyl trichloroacetimidate (i.e., X is —OC(═NH)CCl₃) can be activated by a catalytic amount of a Lewis acid, such as trimethylsilyl triflate or BF₃-etherate. A glycosyl halide (i.e., X is F, Cl, Br or I) is activated by heavy metal ions, mainly mercury or silver. Glycosyl acetates or benzoates (i.e., X is —OAc or —OBz) are preferably first subjected to electrophilic activation to provide a reactive intermediate and then treated with a compound of formula 4. Typical activators of choice are Brønsted acids (e.g., p-TsOH, HClO₄ or sulfamic acid), Lewis acids (e.g., ZnCl₂, SnCl₄, triflate salts, BF₃-etherate, trityl perchlorate, AlCl₃ or triflic anhydride) or a mixture thereof. Pentenyl glycosides (i.e., X is —O—(CH₂)₃—CH═CH₂) can be transglycosylated with a compound of formula 4 in the presence of a promoter such as NBS or NIS. Protic or Lewis acids (triflic acid, Ag-triflate, etc.) can enhance the reaction. Thioglycosides (i.e., X is alkylthio- or optionally substituted phenylthio-group) can be activated by thiophilic promoters such as mercury(II) salts, Br₂, I₂, NBS, NIS, triflic acid, triflate salts, BF₃-etherate, trimethylsilyl triflate, dimethyl-methlythio sulphonium triflate, phenylselenyl triflate, iodonium dicollidine perchlorate, tetrabutylammonium iodide or mixtures thereof, preferably by Br₂, NBS, NIS or triflic acid.

In step b), the R₂ and R₃ protecting groups are removed to provide a compound of formula 2A. A group removable by hydrogenolysis (i.e. optionally substituted benzyl groups) can be deprotected in a catalytic hydrogenolysis reaction (see below). The acyl protecting groups can be removed in a base catalysed transesterification deprotection reaction in an alcoholic solvent such as methanol, ethanol, propanol or t-butanol in the presence of an alcoholate such as NaOMe, NaOEt or KO^{t}Bu.

In a preferred method, a compound of formula 3 wherein R₂ and R₃ are identical and are each benzyl, 4-methoxybenzyl or 4-methylbenzyl, and Y is trichloroacetimidate is coupled with 2,5-dimethyl-4-hydroxy-3-oxo-(2H)-furan followed by catalytic hydrogenolysis.

Acceptors for the Fucosylation/Transfucosylation Reaction

Acceptors of formula H-A-R₁ and salts thereof, wherein A and R₁ are as defined above, can be glycosylated in the enzymatic fucosylation/transfucosylation reaction of this invention.

In a preferred embodiment, the acceptors of formula H-A-R₁ are defucosylated HMOs in anomerically protected form. The most important defucosylated HMOs in anomerically protected form can be selected from the group consisting of lactose, 3′-sialyllactose, 2′-fucosyllactose, 3-fucosyllactose, lacto-N-tetraose (LNT), lacto-N-neotatraose (LNnT), lacto-N-fucopentaose I, lacto-N-fucopentaose II, lacto-N-fucopentaose III, lacto-N-fucopentaose V, LST a (NeuAcα2-3Galβ1-3GlcNAcβ1-3Galβ1-4Glc), LST b (Galβ1-3-[NeuAcα2-6]GlcNAcβ1-3Galβ1-4Glc) and LST c (NeuAcα2-6Galβ1-4GlcNAcβ1-3Galβ1-4Glc), all of them being in 1-O-, 1-S- or 1-N-glycoside form. In a more preferred embodiment, the acceptors are selected from the group consisting of 3′-sialyllactose, 2′-fucosyllactose, 3-fucosyllactose, lacto-N-tetraose (LNT), lacto-N-neotetraose (LNnT), lacto-N-fucopentaose I, lacto-N-fucopentaose II, lacto-N-fucopentaose III, lacto-N-fucopentaose V, LST a (NeuAcα2-3Galβ1-3GlcNAcβ1-3Galβ1-4Glc), LST b (Galβ1-3-[NeuAcα2-6]GlcNAcβ1-3Galβ1-4Glc) and LST c (NeuAcα2-6Galβ1-4GlcNAcβ1-3Galβ1-4Glc), all of them O-glycosylated with a group removable by hydrogenolysis in the anomeric position, preferably with an optionally substituted benzyl or naphthylmethyl group. In a most preferred embodiment, the acceptors are selected from the group consisting of optionally substituted benzyl lactoside, optionally substituted benzyl 3′-sialyllactoside, optionally substituted benzyl 2′-fucosyllactoside, optionally substituted benzyl 3-fucosyllactoside, optionally substituted benzyl LNT-glycoside and optionally substituted benzyl LNnT-glycoside.

The acceptors can be synthesized by treating lactose with acetic anhydride and sodium acetate at a temperature of from 50 to 125° C., followed by a Lewis acid catalyzed glycosylation using R₂—OH or R₃—SH, preferably a benzyl/substituted benzyl alcohol or alkyl-, benzyl- or phenyl-SH in an organic solvent such as DCM, toluene or THF.

Subsequently, the acceptors wherein R₁ is —OR₂ or —SR₃ can be obtained via a final Zemplén deprotection of the glycosylated products.

The acceptors can also be synthesized by treating a fully or partially protected oligosaccharide, preferably a defucosylated HMO with a free anomeric OH, with an R₂ halogenide, preferably a benzyl halogenide or a substituted benzyl halogenide, and sodium hydride, potassium tert-butoxide, potassium carbonate or an inorganic hydroxide in an organic solvent such as DMF, acetonitrile, THF, or dioxane at a temperature of from 0 to 50° C. The anomeric O-protection is followed by removal of the other protecting groups, resulting in acceptors of formula H-A-OR₂.

The vinylogous glycosyl amine acceptors can be synthesized by the treatment of lactose or defucosylated HMO with aqueous ammonium hydrogencarbonate followed by the reaction of the resulting lactosyl amine with an activated vinyl reagent, such as an alkoxymethylenated or dialkylaminomethylenated malonic acid derivative, in the presence or absence of a base (C. Ortiz Mellet et al. J. Carbohydr. Chem. 12, 487 (1993); WO 2007/104311).

Stereo- and Regioselectivity of the Transfucosylation Reaction

A transfucosylation reaction of this invention preferably takes place stereoselectively so that an α-fucosyl bond is formed.

Use of Fucosylated Oligosaccharide Derivatives in the Synthesis of Fucose Containing Oligosaccharides

Another aspect of the present invention is the use of compounds of formula 1 and salts thereof in the synthesis of fucosylated oligosaccharides and salts thereof, the synthesis comprising the step of removing the anomeric protecting group R₁.

In one embodiment, R₁ is —OR₂, wherein R₂ is a protecting group removable by catalytic hydrogenolysis. Removal of the R₂ protecting group typically takes place in a protic solvent or in a mixture of protic solvents. The protic solvent can be selected from the group consisting of water, acetic acid and C₁-C₆ alcohols. A mixture of one or more protic solvents with one or more appropriate aprotic organic solvents miscible partially or fully with the protic solvent(s), such as THF, dioxane, ethyl acetate or acetone, can also be used. Water, one or more C₁-C₆ alcohols or a mixture of water and one or more C₁-C₆ alcohols are preferably used as the solvent system. Solutions or suspensions containing the compounds of formula 1 in any concentration can be used. The reaction mixture is stirred at a temperature of from 10 to 100° C., preferably from 20 to 60° C., in a hydrogen atmosphere of from 1 to 50 bar in the presence of a catalyst such as palladium, Raney nickel or any other appropriate metal catalyst, preferably palladium on charcoal or palladium black, until completion of the reaction. Catalyst concentrations generally range from 0.1% to 10% based on the weight of carbohydrate. Preferably, the catalyst concentrations range from 0.15% to 5%, more preferably 0.25% to 2.25%. Transfer hydrogenolysis can also be carried out, wherein the hydrogen is generated in situ from cyclohexene, cyclohexadiene, formic acid or ammonium formate.

Addition of organic or inorganic bases/acids and/or basic and/or acidic ion exchange resins can also be used to improve the kinetics of the catalytic hydrogenolysis. The use of basic substances is especially preferred when halogen substituents are present on the substituted benzyl moieties of the precursors. Preferred organic bases include triethylamine, diisopropyl ethylamine, ammonia, ammonium carbamate, or diethylamine. Preferred organic/inorganic acids include formic acid, acetic acid, propionic acid, chloroacetic acid, dichloroacetic acid, trifluoroacetic acid, HCl, or HBr.

These conditions allow for the simple, convenient and delicate removal of the anomeric protecting group R₁ to yield pure fucosylated oligosaccharides which can be isolated from the reaction mixture using conventional work-up procedures in crystalline, amorphous solid, syrupy form or in a concentrated aqueous solution.

In another embodiment, R₁ is —SR₃ wherein R₃ is optionally substituted alkyl, optionally substituted aryl or optionally substituted benzyl, which compounds can be converted into the corresponding reducing sugar derivatives in the following way: the thioglycoside of formula 1 is dissolved in water or a dipolar aprotic solvent containing water followed by the addition of a thiophilic activator such as mercury(II) salts, Br₂, I₂, NBS, NIS, triflic acid or triflate salts, or a mixture thereof. The activated intermediate reacts easily with the water present in the reaction milieu and a deprotected fucooligosaccharide is produced.

In another embodiment, a fucosylated oligosaccharide can be formed by removal of an acyclic vinylogous amine group from a compound of formula 1, wherein R₁ is —NH—C(R″)═C(R′)₂, and R′ is an electron withdrawing group selected from the group consisting of —CN, —COOH, —COO-alkyl, —CO-alkyl, —CONH₂, —CONH-alkyl, —CONH-benzyl, —CON(alkyl)₂ and —CON(benzyl)₂, or two R′-groups linked together form —CO—(CH₂)₂₋₄—CO—, thus forming with the adjacent carbon atom a 5-7 membered cycloalkan-1,3-dione, and any of the methylene groups can be substituted with 1 or 2 alkyl groups; and R″ is H or alkyl. The enamine structure can be split by treatment with amino compounds or a halogen. Solvents for the reaction include methanol, ethanol, water, acetic acid, or ethyl acetate, and mixtures thereof. Amino compounds for the reaction are the aqueous and anhydrous primary amines, such as ethylamine, propylamine and butylamine, the hydrazines, such as hydrazine hydrate and hydrazine acetate, hydroxylamine derivatives, an aqueous ammonia solution and ammonia gas. The acyclic vinylogous amine can also be cleaved with a halogen such as chlorine gas or bromine. Both types of reactions yield amine functionality at the anomeric position, the hydrolysis of which under neutral or slightly acidic pH (pH≈4-7) readily provides a fucosylated oligosaccharide.

Preferably, the compound of formula 1 is a compound of formula 1′

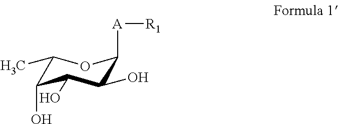

wherein A is a carbohydrate linker which is either a lactosyl moiety or contsists of a lactosyl moiety and at least one monosaccharide unit selected from the group consisting of: glucose, galactose, N-acetylglucosamine, fucose and N-acetyl neuraminic acid; and wherein R₁ is one of the following anomeric protecting groups:
   a) —OR₂, wherein R₂ is a protecting group removable by catalytic hydrogenolysis,
   b) —SR₃, wherein R₃ is an optionally substituted alkyl, an optionally substituted aryl or an optionally substituted benzyl,
   c) —NH—C(R″)═C(R′)₂, wherein each R′ independently is one of the following electron withdrawing groups: —CN, —COOH, —COO-alkyl, —CO-alkyl, —CONH₂, —CONH-alkyl or —CON(alkyl)₂, or wherein the two R′-groups are linked together and form —CO—(CH₂)₂₋₄—CO— and thus form, together with the carbon atom to which they are attached, a 5-7 membered cycloalkan-1,3-dione, in which dione any of the methylene groups is optionally substituted with 1 or 2 alkyl groups, and R″ is H or alkyl,
   provided that if R₁ is —OR₂ then linker A does not comprise N-acetyl neuraminic acid.

Fucose-containing human milk oligosaccharides can easily be produced by removing the anomeric protecting group of the novel fucose-containing HMOs β-R₁-glycosides of the present invention according to the procedures specified above. Other features of the invention will become apparent in the course of the following descriptions of exemplary embodiments which are given for illustration of the invention and are not to be limiting thereof.

### EXAMPLES

### Example 1

### Manufacture of Fucosyl Acceptors

A) General procedure: lactose (5 g, 14.6 mmol) and TsOH.H₂O (0.2 g, 1.05 mmol) were added in one portion to a mixture of DMF (20 ml) and benzaldehyde dimethyl acetal (5.5 ml, 35.4 mmol, 2.4 eq.) at room temperature. The reaction mixture was vigorously stirred at 70° C. under exclusion of humidity for 1 hour. After cooling triethyl amine (0.15 ml) was added then the volatile components (MeOH, triethyl amine, remaining benzaldehyde dimethyl acetal) were removed in vacuo. To the reaction mixture the benzyl bromide derivative (1.5 eq.)—predissolved in 5-10 ml of DMF, if the reagent is a solid—was added and the mixture was cooled to 0° C. for 20 min. Still under cooling NaH (0.8 g of a 55% dispersion in mineral oil, 1.3 eq.) was added in one portion, and the mixture was stirred under cooling until the hydrogen formation stopped then at room temperature for 2-3 hours. Methanol (2 ml) was added carefully and the reaction was stirred for a further 5 min. The reaction mixture was portioned between 100 ml of DCM and 100 ml of water and extracted. The water layer was back-extracted twice with 100 ml of DCM. The combined organic phases were evaporated; the residue was dissolved in 100 ml of acetonitrile and extracted with 100 ml of hexane. The acetonitrile was distilled off and the residue was taken up in isopropanol (10 ml) and isopropyl ether (50 ml) at 50° C. The clear solution was cooled to −20° C. for between 2-12 hours. The crystals obtained were filtered off and washed twice with TBME and dried. Recrystallization can be carried out from a mixture of TBME (˜50 ml) and ethanol (˜20 ml).

4-Chlorobenzyl 4′,6′-O-benzylidene-β-lactoside

Yield: 1.71 g

4-Methylbenzyl 4′,6′-O-benzylidene-β-lactoside

Yield: 3.20 g

3-Phenylbenzyl 4′,6′-O-benzylidene-β-lactoside

Yield: 2.70 g

2-Naphthylmethyl 4′,6′-O-benzylidene-β-lactoside

Yield: 1.77 g

B) To a mixture of one of the above benzylidene acetals (500 mg) in methanol (10 ml) and water (0.5 ml) TFA was added at room temperature and the reaction mixture was stirred for 2-4 hours under exclusion of humidity then evaporated. The remaining material was co-evaporated with ethanol 3-4 times giving a crude solid, which, after drying, can be recrystallized from a mixture of methanol (˜10-35 ml) and water (—O—2 mL).

4-Chlorobenzyl β-lactoside

Yield: 333 mg

¹³C-NMR (75.1 MHz, D₂O): δ=135.25, 133.67, 130.30, 128.70, 103.00, 101.13, 78.39, 75.44, 74.89, 74.49, 72.88, 72.58, 71.03, 70.83, 68.62, 61.11, 60.13.

4-Methylbenzyl β-lactoside

Yield: 439 mg

¹³C-NMR (75.1 MHz, D₂O): δ=138.91, 133.50, 129.37, 129.07, 103.01, 100.96, 78.43, 75.44, 74.87, 74.52, 72.90, 72.59, 71.47, 71.03, 68.63, 61.11, 60.17, 20.34.

3-Phenylbenzyl β-lactoside

Yield: 438 mg

¹³C-NMR (75.1 MHz, d₆-DMSO/d₄-MeOH/D₂O 8:1:1): δ=140.29, 140.24, 138.88, 129.13, 129.02, 127.66, 126.88, 126.83, 126.03, 125.90, 103.95, 102.03, 80.76, 75.65, 75.07, 75.00, 73.34, 73.28, 70.66, 69.81, 68.27, 60.56.

2-Naphthylmethyl β-lactoside

Yield: 378 mg

¹³C-NMR (75.1 MHz, D₂O/d₆-DMSO): δ=134.96, 133.24, 133.12, 128.59, 128.31, 128.08, 127.46, 126.98, 126.90, 126.79, 103.26, 101.59, 78.89, 75.62, 75.09, 74.81, 73.14, 72.81, 71.33, 71.14, 68.75, 61.22, 60.39.

C)

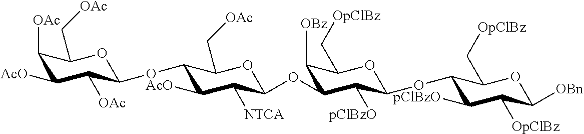

10 g (8.13 mmol) of benzyl 2,3,6,2′,6′-penta-O-(4-chlorobenzoyl)-4′-O-benzoyl-β-lactoside and 10 g (1.6 equiv.) of methyl N-trichloroacetyl-3,6,2′,3′,4ζ6′-hexa-O-acetyl-1-thio-lactosaminide were dissolved in 35 ml of dry CHCl₃ under argon. To this solution 3.7 g of NIS and 490 mg of AgOTf were added at room temperature, and the stirring was continued for approx. 20 min. Triethyl amine (5 ml) was added to the slurry, diluted with CH₂Cl₂ (500 ml) and then extracted 2× with sodium thiosulphate solution (10%), the organic phase was separated, dried with MgSO₄, filtered, concentrated, and the syrup was chromatographed on a column of silica-gel, using a gradient of CH₂Cl₂: acetone 98:2→95:5. Yield: 12.7 g, 80%. MS (ESP): 1972.1 [M+Na]⁺, 1988.1 [M+K]⁺, 1948.2 [M−H]⁻, 1984.0 [M+Cl]⁻. ¹³C NMR (CDCl₃) δ: 101.2, 100.7, 100.0, 98.8 (anomeric carbons).

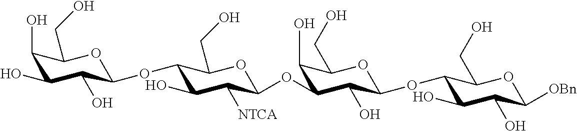

10 g (5.1 mmol) of the tetrasaccharide prepared above was dissolved in MeOH (110 ml) and a solution of NaOMe (1M in MeOH) was added until pH 10 was attained. The solution was stirred at 40° C. for 5 h, then was neutralized by addition of Amberlite IR 120 H⁺ resin, the resin was filtered off, and the filtrate was evaporated to dryness. The residue was dissolved in warm DMF (10 ml) and added dropwise to ⁱPr₂O (150 ml) and the suspension was stirred for an additional 3 h. The precipitate was filtered off, washed with ⁱPr₂O (2×20 ml) and dried to yield 4.2 g of product as off-white powder (91%). MS (ESP): 900.1 [M−H]⁻. ¹³C NMR (D₂O) δ: 105.6, 105.5, 104.2, 103.7 (anomeric carbons).

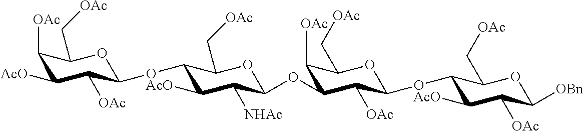

35 g of a compound of the tetrasaccharide prepared above was dissolved in 110 ml of MeOH and 110 ml of aqueous KOH (7.5 g) solution and the mixture was stirred at rt. for Id. The mixture was then chilled with an ice-bath, neutralized by HCl-gas and concentrated to dryness. The resulting crude brown glass was then acetylated with pyridine (150 ml) and acetic anhydride (150 ml) at rt. for 1 d. The solution was concentrated, the syrup was dissolved in CH₂Cl₂, the organic phase was extracted with 1M HCl-solution and then with sat. NaHCO₃-solution, dried with MgSO₄, filtered and concentrated to yield 43 g of brown foam, which was subjected to column chromatography using CH₂Cl₂:acetone 8:2 as eluent. ¹³C NMR (CDCl₃) δ: 101.2, 100.8, 100.4, 99.2 (anomeric carbons).

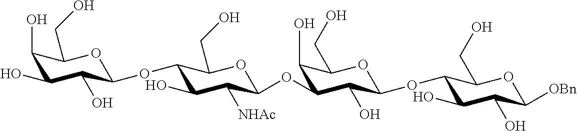

140 g (107.5 mmol) of the peracetylated tetrasaccharide prepared above was dissolved in 1.5 L of MeOH, NaOMe-solution (1M) was added until pH 10, and the mixture was stirred at 50° C. overnight. The product crystallized from the reaction mixture. The mixture is allowed to cool to rt., then it was chilled, filtered, the filtrate was washed with cold EtOH, then dried to yield 69 g of benzyl β-LNnT as a white powder (86.5 mmol, 80%). ¹³C NMR (D₂O) δ: 105.6, 105.5, 105.4, 103.6 (anomeric carbons). Mp. 284-286° C.

D) Phenyl 1-thio-P-lactoside and benzyl 1-thio-P-lactoside were prepared according to the procedure described by Y. Nagao et al. Chem. Pharm. Bull. 43, 1536 (1995) and the characterizations were identical with those reported.

E) Galβ1-3GlcNAcβ1-3Galβ1-4Glcβ1-O-Bn (1-O-benzyl-β-LNT) can be prepared according to A. Malleron et al. Carbohydr. Res. 341, 29 (2006).

### Example 2

### Manufacture of a Fucosyl Donor of Formula 2A

a) The solution of 2,3,4-tri-O-(4-methoxybenzyl)-L-fucopyranose trichloroacetimidate (a/(3 mixture, prepared from 85 mmol of 2,3,4-tri-O-(4-methoxybenzyl)-L-fucopyranose and trichloroacetonitrile in the presence of NaH in quantitative yield) in diethyl ether (100 ml) was added to a mixture of 2,5-dimethyl-4-hydroxy-3-oxo-(2H)-furan (85 mmol) and TMS-triflate (1.2 ml) in diethyl ether (200 ml) at −14° C. After 3 hours the cooling bath was removed and the stirring continued for 1 hour. The reaction mixture was diluted with ethyl acetate and extracted with sat. NaHCO₃-solution (3×150 ml) and brine (150 ml). The organic phase was dried over Na₂SO₄ and evaporated. The resulting syrup was purified by column chromatography yielding 23.27 g of 2,5-dimethyl-3-oxo-(2H)-furan-4-yl 2,3,4-tri-O-(4-methoxybenzyl)-α-L-fucopyranoside as a thick yellow syrup in a 1:1 mixture of diastereoisomers. Selected NMR chemical shifts in CDCl₃:anomeric protons:5.32 and 5.57 ppm, J_{1,2}=3.4 Hz; anomeric carbons: 113.81 and 113.95 Hz.
b) A suspension of the above product and Pd on charcoal (12.5 g) in ethyl acetate (1 1) was stirred at room temperature for 6 hours under H₂. The catalyst was filtered off and washed with ethyl acetate. The combined filtrate was evaporated to a solid mass which was suspended in ethyl acetate and allowed to stand at 5° C. for overnight. The crystalline material was filtered off and dried. The mother liquor was subjected to column chromatography. The combined products gave 5.50 g of 2,5-dimethyl-3-oxo-(2H)-furan-4-yl α-L-fucopyranoside as white solid in 1:1 mixture of diastereoisomers. Selected NMR chemical shifts in DMSO-d₆: anomeric protons: 5.07 and 5.52 ppm, J_{1,2}=2.6 Hz; anomeric carbons: 100.19 and 101.03 Hz. Purity by GC (after silylation): 98.9%.

### Example 3

### Transfucosylation Reactions

General procedure: A solution of the appropriate fucosyl donor (such as p-nitrophenyl α-L-fucopyranoside, α-L-fucosyl fluoride, 2,5-dimethyl-3-oxo-(2H)-furan-4-yl α-L-fucopyranoside or 2′-O-fucosyllactose) and acceptor (10-500 mmol, donor acceptor ratio is 5:1 to 1:5) was incubated in degassed incubation buffer at a pH range from 5.0 to 9.0) with recombinant α-fucosidase, α-transfucosidase or α-fucosynthase. The reaction mixture was stirred for 24 hours at a temperature range from 20 to 70° C.

Samples were taken at different times of the reaction, the reaction was stopped by the addition of 1M NaHCO₃-solution at pH=10 and analyzed by TLC and/or HPLC. After completion, the enzyme was denatured and centrifuged. The resulting solution was evaporated under reduced pressure. After lyophilisation, the dry residue was dissolved in water and purified by biogel chromatography (P-2 Biogel, 16×900 mm) with water or by reverse phase chromatography. The yields vary between 2.5-85%.

Recombinant enzymes used and tested in transfucosylation reaction:

P25 from Thermotoga Maritima (see seq. ID 1) containing mutations G226S Y237H T264A L322P;
M3 from Thermotoga Maritima (see seq. ID 1) containing mutations Y237H Y267F L322P.

These transfucosidases were produced in E. coli as reported in G. Osanjo et al. Biochemistry 46, 1022 (2007). Purified transfucosidases P25 and M3 are stored at −20° C. or +4° C., respectively.

Optimal buffer for the enzymes: 50 mM sodium citrate/phosphate buffer and 150 mM NaCl, pH=5.5.

Optimum temperature: 60° C. (except for α-L-fucosyl fluoride acceptor, where the temperature is 35° C.).

LC-MS conditions:
Instrument: AB Sciex API 2000 tandem MS
Ionization mode: electrospray in positive mode
Scan type: Q1MS
Sample insertion mode: HPLC
Column: Phenomenex HILIC 250×4.6 mm
Flow: isocratic (water-acetonitrile 22:78)
Flow rate: 1 ml/min
Injected volume: 5 μl
   a) acceptor: Galβ1-4Glcpl-O-Bn
donor: p-nitrophenyl α-L-fucopyranoside, α-L-fucosyl fluoride or 2,5-dimethyl-3-oxo-(2H)-furan-4-yl α-L-fucopyranoside
product: Fucα1-2Galβ1-4Glcpl-O-Bn (identified by comparison to the standard sample made by chemical means from 2′-O-fucosyllactose)
Characteristic ¹H NMR peaks (DMSO-d₆) δ: 7.41-7.25 (m, 5H, aromatic), 5.20 (d, 1H, J_{1″,2″}=2 Hz, H-1″), 4.82 and 4.59 (ABq, 2H, J_{gem}=12.3 Hz, —CH₂Ph), 4.32 (d, 1H, J_{1′,2′}=7.31 Hz, H-1′), 4.28 (d, 1H, J_{1,2}=7.79 Hz, H-1), 1.05 (d, 1H, J_{5″,6″}=6.43 Hz, H-6″).
¹³C NMR (DMSO-d₆) δ: 137.97, 128.15, 128.15, 127.58, 127.58 and 127.43 (aromatic), 101.86, 100.94 and 100.20 (C-1, C-1′ and C-1″), 77.68, 76.78, 75.36, 75.33, 74.70, 73.79, 73.45, 71.60, 69.72, 69.69, 68.74, 68.20, 66.38, 60.23 and 59.81 (C-2, C-3, C-4, C-5, C-6, C-2′, C-3′, C-4′, C-5′, C-6′, C-2″, C-3″, C-4″, C-5″ and CH₂Ph), 16.47 (C-6″).
   b) acceptor: Galβ1-4Glc31-O—CH₂-(4-NO₂-Ph)
donor: p-nitrophenyl α-L-fucopyranoside or 2,5-dimethyl-3-oxo-(2H)-furan-4-yl α-L-fucopyranoside
product: Fucα1-2Galβ1-4Glcβ1-O—CH₂-(4-NO₂-Ph) (identified by comparison to the standard sample made by chemical means from 2′-O-fucosyllactose)
Characteristic ¹H NMR peaks (DMSO-d₆) δ: 8.20 and 7.68 (each d, 4H, aromatic), 5.04 (d, 1H, J_{1″,2″}=2 Hz, H-1″), 4.97 and 4.76 (ABq, 2H, J_{gem}=12.3 Hz, —CH₂Ph), 4.40 (d, 1H, J_{1′,2′}=9.53 Hz, H-1′), 4.32 (d, 1H, J_{1,2}=8.04 Hz, H-1), 1.04 (d, 1H, J_{5″,6″}=6.43 Hz, H-6″).
¹³C NMR (DMSO-d₆) δ: 162.38, 147.68, 127.95, 127.95, 123.33 and 123.33 (aromatic), 102.18, 100.95 and 100.18 (C-1, C-1′ and C-1″), 77.62, 76.74, 75.36, 75.36, 74.61, 73.78, 73.45, 71.59, 69.67, 68.74, 68.67, 68.21, 66.38, 60.22 and 59.77 (C-2, C-3, C-4, C-5, C-6, C-2′, C-3′, C-4′, C-5′, C-6′, C-2″, C-3″, C-4″, C-5″ and CH₂Ph), 16.45 (C-6″).
   c) acceptor: Galβ1-4Glcβ1-S-Bn
donor: p-nitrophenyl α-L-fucopyranoside or 2,5-dimethyl-3-oxo-(2H)-furan-4-yl α-L-fucopyranoside
product: Fucα1-2Galβ1-4Glcβ1-S-Bn (identified by comparison to the standard sample made by chemical means from 2′-O-fucosyllactose)
Characteristic ¹H NMR peaks (DMSO-d₆) δ: 7.36-7.20 (m, 5H, aromatic), 5.00 (bs, 1H, H-1″), 4.34 (d, 1H, J_{1,2}=6.80 Hz, H-1′), 3.98 (d, 1H, J_{1,2}=9.33 Hz, H-1), 3.94 and 3.78 (ABq, 2H, J_{gem}=12.77 Hz, —CH₂Ph), 1.05 (d, 1H, J_{5″,6″}=6.47 Hz, H-6″).
¹³C NMR (DMSO-d₆) δ: 138.12, 129.11, 129.12, 128.29, 128.29 and 124.84 (aromatic), 100.90 and 100.39 (C-1′ and C-1″) 82.74 (C-1), 79.44, 77.85, 77.05, 76.27, 75.38, 73.74, 72.94, 71.63, 69.72, 68.78, 68.18, 68.38, 60.24 and 60.10 (C-2, C-3, C-4, C-5, C-6, C-2′, C-3′, C-4′, C-5′, C-6′, C-2″, C-3″, C-4″, C-5″), 32.29 (—CH₂Ph), 16.47 (C-6″).
   d) acceptor: Galβ1-4Glcβ1-S-Ph
donor: p-nitrophenyl α-L-fucopyranoside or 2,5-dimethyl-3-oxo-(2H)-furan-4-yl α-L-fucopyranoside
product: Fucα1-2Galβ1-4Glcβ1-S-Ph (identified by comparison to the standard sample made by chemical means from 2′-O-fucosyllactose)
Characteristic ¹H NMR peaks (DMSO-d₆) δ: 7.50-7.46 and 7.38-7.22 (m, 5H, aromatic), 5.02 (d, 1H, J_{1″,2″}=2.21 Hz, H-1″), 4.66 (d, 1H, J_{1,2}=9.78 Hz, H-1), 4.35 (d, 1H, J_{1′,2′}=7.41 Hz, H-1″), 1.05 (d, 1H, J_{5″,6″}=6.46 Hz, H-6″).
¹³C NMR (DMSO-d₆) δ: 134.20, 130.15, 130.15, 128.86, 128.86 and 126.59 (aromatic), 100.84 and 100.12 (C-1′ and C-1″), 86.35 (C-1), 79.06, 76.99, 76.70, 75.84, 75.32, 73.68, 72.22, 71.45, 69.54, 68.65, 68.05, 66.38, 60.12 and 59.54 (C-2, C-3, C-4, C-5, C-6, C-2′, C-3′, C-4′, C-5′, C-6′, C-2″, C-3″, C-4″, C-5″), 16.44 (C-6″).
   e) acceptor: Galβ1-3GlcNAcβ1-3Galβ1-4Glcβ1-O-Bn
donor: α-L-fucosyl fluoride, 2′-O-fucosyllactose or 2,5-dimethyl-3-oxo-(2H)-furan-4-yl α-L-fucopyranoside
product: monofucosylated Galβ1-3GlcNAcβ1-3Galβ1-4Glcβ1-O-Bn, correct molecular mass was confirmed by LC-MS (944 [M+H]⁺, 961 [M+NH₄]⁺, 966 [M+Na]⁺)
   f) acceptor: Galβ1-4GlcNAcβ1-3Galβ1-4Glcβ1-O-Bn
donor: α-L-fucosyl fluoride, 2′-O-fucosyllactose or 2,5-dimethyl-3-oxo-(2H)-furan-4-yl α-L-fucopyranoside
product: monofucosylated Galβ1-4GlcNAcβ1-3Galβ1-4Glcβ1-O-Bn, correct molecular mass was confirmed by LC-MS (944 [M+H]⁺, 961 [M+NH₄]⁺, 966 [M+Na]⁺)

Having thus described the invention with reference to preferred embodiments, it will be appreciated that various modifications are possible within the scope of the invention.

In this specification, unless expressly otherwise indicated, the word ‘or’ is used in the sense of an operator that returns a true value when either or both of the stated conditions is met, as opposed to the operator ‘exclusive or’ which requires that only one of the conditions is met. The word ‘comprising’ is used in the sense of ‘including’ rather than in to mean ‘consisting of’. All prior teachings acknowledged above are hereby incorporated by reference. No acknowledgement of any prior published document herein should be taken to be an admission or representation that the teaching thereof was common general knowledge in Australia or elsewhere at the date hereof.

1. A method for the synthesis of a compound of formula 1 or a salt thereof, 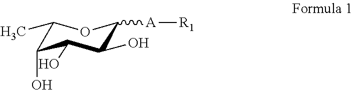
wherein A is a carbohydrate linker which is a lactosyl moiety or which consists of a lactosyl moiety and at least one monosaccharide unit selected from the group consisting of: glucose, galactose, N-acetylglucosamine, fucose and N-acetyl neuraminic acid; and wherein R₁ is one of the following anomeric protecting groups:
a) —OR₂, wherein R₂ is a protecting group removable by catalytic hydrogenolysis,
b) —SR₃, wherein R₃ is an optionally substituted alkyl, an optionally substituted aryl or an optionally substituted benzyl,
c) —NH—C(R″)═C(R′)₂, wherein each R′ independently is one of the following electron withdrawing groups: —CN, —COOH, —COO-alkyl, —CO-alkyl, —CONH₂, —CONH-alkyl or —CON(alkyl)₂, or wherein the two R′-groups are linked together and form —CO—(CH₂)₂₋₄—CO— and thus form, together with the carbon atom to which they are attached, a 5-7 membered cycloalkan-1,3-dione, in which dione any of the methylene groups is optionally substituted with 1 or 2 alkyl groups, and R″ is H or alkyl,
characterized in that a fucosyl donor of formula 2 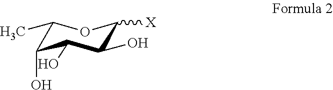
wherein X is selected from the group consisting of: a guanosine diphosphatyl moiety, a lactose moiety, azide, fluoride, optionally substituted phenoxy-, optionally substituted pyridinyloxy-, optionally substituted 3-oxo-furanyloxy- of formula A, optionally substituted 1,3,5-triazinyloxy- of formula B, 4-methylumbelliferyloxy-group of formula C, and a group of formula D 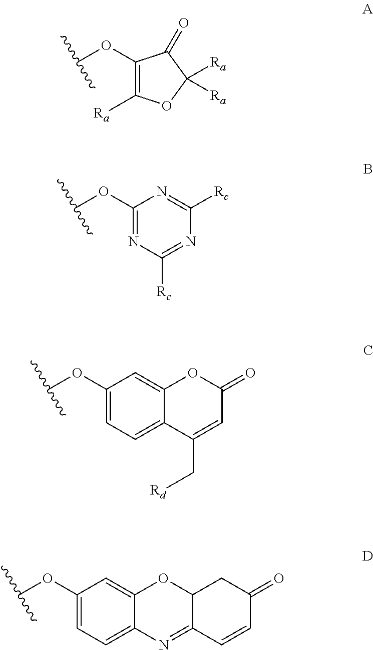
wherein Rₐ is independently H or alkyl, or two vicinal Rₐ groups represent a ═C(R_{b})₂ group, wherein R_{b} is independently H or alkyl, R_{c} is independently selected from the group consisting of alkoxy, amino, alkylamino and dialkylamino, R_{d} is selected from the group consisting of H, alkyl and —C(═O)Rₑ, wherein Rₑ is OH, alkoxy, amino, alkylamino, dialkylamino, hydrazino, alkylhydrazino, dialkylhydrazino or trialkylhydrazino, is reacted with an acceptor of formula H-A-R₁ or a salt thereof, wherein A and R₁ are as defined above, under the catalysis of an enzyme capable of transferring fucose.

2. The method according to claim 1, wherein the enzyme is a fucosyltransferase or a fucosidase.

3. The method according to claim 2, wherein the fucosidase is an engineered transfucosidase or an engineered fucosynthase.

4. The method according to claim 3, wherein the engineered transfucosidase or the engineered fucosynthase stems from Bifidobacterium bifidum, Sulfolobus solfataricus or Thermotoga maritima.

5. The method according to claim 2, wherein the fucosidase is an engineered α-transfucosidase, and wherein either the compound of formula 2 is 2′-O-fucosyllactose, or X in formula 2 is fluoride, phenoxy-, p-nitrophenoxy-, 2,4-dinitrophenoxy-, 2-chloro-4-nitrophenoxy-, 4,6-dimethoxy-1,3,5-triazin-2-yloxy-, 4,6-diethoxy-1,3,5-triazin-2-yloxy-, 2-ethyl-5-methyl-3-oxo-(2H)-furan-4-yloxy-, 5-ethyl-2-methyl-3-oxo-(2H)-furan-4-yloxy- or 2,5-dimethyl-3-oxo-(2H)-furan-4-yloxy-group.

6. The method according to claim 5, wherein the acceptor is a defucosylated human milk oligosaccharide in anomerically protected form.

7. A compound of formula 1 or a salt thereof, 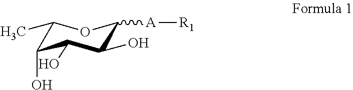
wherein A is a carbohydrate linker which is a lactosyl moiety or which consists of a lactosyl moiety and at least one monosaccharide unit selected from the group consisting of: glucose, galactose, N-acetylglucosamine, fucose and N-acetyl neuraminic acid, and wherein R₁ is one of the following anomeric protecting groups:
a) —OR₂, wherein R₂ is a protecting group removable by catalytic hydrogenolysis,
b) —SR₃, wherein R₃ is an optionally substituted alkyl, an optionally substituted aryl or an optionally substituted benzyl,
c) —NH—C(R″)═C(R′)₂, wherein each R′ independently is one of the following electron withdrawing groups: —CN, —COOH, —COO-alkyl, —CO-alkyl, —CONH₂, CONH-alkyl or —CON(alkyl)₂, or wherein the two R′-groups are linked together and form —CO—(CH₂)₂₋₄—CO— and thus form, together with the carbon atom to which they are attached, a 5-7 membered cycloalkan-1,3-dione, in which dione any of the methylene groups is optionally substituted with 1 or 2 alkyl groups, and R″ is H or alkyl,
provided that if R₁ is —OR₂ then linker A does not comprise N-acetyl neuraminic acid.

8. The compound according to claim 7 characterized by formula 1′ 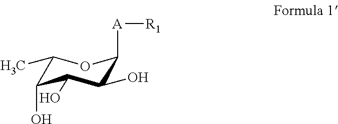
wherein A and R₁ are as defined in claim 7.

9. The compound according to claim 8, wherein A together with the terminal fucosyl moiety is a human milk oligosaccharide glycosyl residue.

10. The compound according to claim 9, wherein A comprises a lactosaminyl residue and/or an isolactosaminyl residue.

11. The compound according to claim 8 selected from the group consisting of β-R₁-glycosides of: 2′-O-fucosyllactose, 3-O-fucosyllactose, 3′-O-sialyl-3-O-fucosyl-lactose, difucosyllactose, lacto-N-fucopentaose I, lacto-N-fucopentaose II, lacto-N-fucopentaose III, lacto-N-fucopentaose V, lacto-N-difuco-hexaose I, lacto-N-difuco-hexaose II, lacto-N-difuco-hexaose III, F-LST a, F-LST b and F-LST c.

12. The compound according to claim 11 selected from the group consisting of β-OR₂- and β-SR₃-glycosides of: 2′-O-fucosyllactose, 3-O-fucosyllactose, difucosyllactose, lacto-N-fucopentaose I, lacto-N-fucopentaose II, lacto-N-fucopentaose III, lacto-N-fucopentaose V, F-LST a, F-LST b and F-LST c.

13. The compound according to claim 12, wherein R₂ is a benzyl or 2-naphthylmethyl group, each of which is optionally substituted with at least one group selected from the group consisting of phenyl, alkyl or halogen, or wherein R₃ is phenyl or benzyl.

14. (canceled)

15. (canceled)

16. A method of manufacture of a human milk oligosaccharide or a salt thereof, comprising the step of removing the anomeric protecting group R₁ from a compound of formula 1′ or a salt thereof 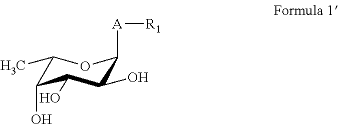
wherein A is a carbohydrate linker which is a lactosyl moiety or which consists of a lactosyl moiety and at least one monosaccharide unit selected from the group consisting of: glucose, galactose, N-acetylglucosamine, fucose and N-acetyl neuraminic acid; and wherein R₁ is one of the following anomeric protecting groups:
a) —OR₂, wherein R₂ is a protecting group removable by catalytic hydrogenolysis,
b) —SR₃, wherein R₃ is an optionally substituted alkyl, an optionally substituted aryl or an optionally substituted benzyl,
c) —NH—C(R″)═C(R′)₂, wherein each R′ independently is one of the following electron withdrawing groups: —CN, —COOH, —COO-alkyl, —CO-alkyl, —CONH₂, —CONH-alkyl or —CON(alkyl)₂, or wherein the two R′-groups are linked together and form —CO—(CH₂)₂₋₄—CO— and thus form, together with the carbon atom to which they are attached, a 5-7 membered cycloalkan-1,3-dione, in which dione any of the methylene groups is optionally substituted with 1 or 2 alkyl groups, and R″ is H or alkyl,
provided that if R₁ is —OR₂ then linker A does not comprise N-acetyl neuraminic acid.

17. The method of claim 16, wherein the compound of formula 1′ or the salt thereof is formed by reacting a fucosyl donor of formula 2 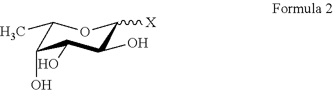
wherein X is selected from the group consisting of: a guanosine diphosphatyl moiety, a lactose moiety, azide, fluoride, optionally substituted phenoxy-, optionally substituted pyridinyloxy-, optionally substituted 3-oxo-furanyloxy- of formula A, optionally substituted 1,3,5-triazinyloxy- of formula B, 4-methylumbelliferyloxy-group of formula C, and a group of formula D 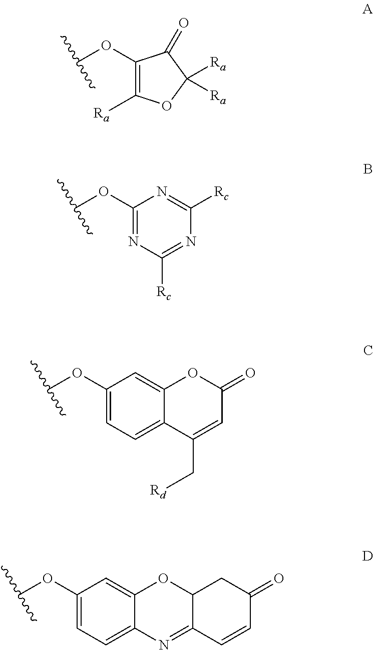
wherein R₁ is independently H or alkyl, or two vicinal Rₐ groups represent a ═C(R_{b})₂ group, wherein R_{b} is independently H or alkyl, R₁ is independently selected from the group consisting of alkoxy, amino, alkylamino and dialkylamino, R_{d} is selected from the group consisting of H, alkyl and —C(═O)Rₑ, wherein R₁ is OH, alkoxy, amino, alkylamino, dialkylamino, hydrazino, alkylhydrazino, dialkylhydrazino or trialkylhydrazino,
with an acceptor of formula H-A-R₁ or a salt thereof, under the catalysis of an enzyme capable of transferring fucose.

18. A method of manufacture of a fucosylated oligosaccharide or a salt thereof, comprising the steps of:
synthesis of a compound of formula 1 or a salt thereof in accordance with claim 1, and
removing the anomeric protecting group R₁ from the compound of formula 1 or a salt thereof.

19. A compound of formula 2A 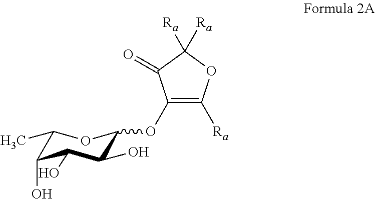
wherein Rₐ is independently H or alkyl, or two vicinal Rₐ groups represent a ═C(R_{b})₂ group, wherein R_{b} is independently H or alkyl, and preferably wherein Rₐ is independently H, methyl or ethyl.
